(19) United States
(12) Patent Application Publication
(10) Pub. No.: US 2026/0263703 A1
(43) Pub. Date: Sep. 10, 2026

(54) INJECTION SYSTEMS AND METHODS

(71) Applicant: CREDENCE MEDSYSTEMS, INC., Menlo Park, CA (US)

(72) Inventors: Jeff Tillack, Wake Forest, NC (US); Allyson Jade Weiss, San Jose, CA (US); Alan E. Shluzas, San Carlos, CA (US)

(73) Assignee: CREDENCE MEDSYSTEMS, INC., Menlo Park, CA (US)

(21) Appl. No.: 19/375,994

(22) Filed: Oct. 31, 2025

Related U.S. Application Data
(60) Provisional application No. 63/715,452, filed on Nov. 1, 2024.

(51) Int. Cl. A61M 5/32

(52) U.S. Cl. CPC A61M 5/3202; A61M 2205/27; A61M 2205/8281

(57) ABSTRACT

An injection system includes a housing member having a housing body defining a housing interior and an injection member disposed in the housing interior. The injection member includes a syringe body having proximal and distal ends and defining a syringe interior. The injection member also includes a stopper member disposed in the syringe interior, and a plunger member coupled to the stopper member and configured to be manipulated to insert the stopper member distally in the syringe interior relative to the syringe body. The injection member further includes a needle hub assembly coupled to the syringe body at the distal end thereof. The needle hub assembly includes a needle hub and a needle coupled to the needle hub. The housing member includes a needle obscuring member slidably coupled to the housing body. The needle obscuring member is configured to be disposed at least partially over the needle.

The present application claims priority to U.S. Provisional Patent Application Ser. No. 63/715,452, filed on Nov. 1, 2024 under attorney docket number CM.30047.00 and entitled “INJECTION SYSTEMS AND METHODS.” The present application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”; (16) U.S. patent application Ser. No. 18/300,125, filed on Apr. 13, 2023 under attorney docket number CM.20035.00 and entitled “NEEDLE SHIELD MANAGEMENT AND NEEDLE HIDING DEVICES FOR INJECTION SYSTEMS AND METHODS”; and U.S. Provisional Patent Application Ser. No. 63/596,404, filed on Nov. 6, 2023 under attorney docket number CM.30043.00 and entitled “NEEDLE SHIELD MANAGEMENT AND NEEDLE OBSCURING DEVICES FOR INJECTION SYSTEMS AND METHODS.” The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to injection systems with housings, and methods related to obscuring, concealing, or hiding a needle in such injection systems, with or without safety features and/or needle shield handling devices, in the home and/or in out of home healthcare environments. These devices are intended to be used by a device user who will be administering an injection to a patient. The device user may be a healthcare professional, nurse, doctor, surgeon, clinician, and/or a person who gives the injection to the patient. Alternatively, the device user may be a patient who will administer the injection to themselves (self-injector).

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A to 4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and visually obscuring, concealing, or hiding the needle in injection systems. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3 to 4B) and relatively high inventory and utilization expenses.

Most injection systems include a needle shield (e.g., rigid needle shield, needle shield, etc.) removably coupled to a needle hub and configured to cover the sharp distal end of the needle to minimize accidental needle sticks, to protect the needle from mechanical damage, and to seal the injection system before use. Existing injection systems are configured such that needle shields are removed manually by a user's hands. Needle shields are secured to needle hubs. Significant force must be exerted by the user's hands to remove needle shields from needle hubs. The user's hands are proximate the sharp distal end of the needle when removing the needle shield. As a result of this proximity, the removal of the needle shield can result in accidental needle sticks (e.g., with a bounce back of the hand grasping the needle shield or movement of the hand holding the injection system). When a user applies force to the proximal end of the needle shield to avoid needle sticks, the needle shield may be launched from the needle hub and end up as unwanted trash on the floor of the medical facility. Accordingly, there exists a need for needle shield handling devices for a more controlled and safer removal of needle shields from injection systems.

Further, for multiple chamber injection systems such as those described in U.S. patent application Ser. No. 15/801,259, which was previously incorporated by reference herein, pressure may build up in a distal chamber when mixing drug components therein prior to injection with the distal end of the needle sealed by a needle shield. Additionally, the needle may be fixedly or removably mounted to a flange on a cartridge body instead of a syringe. Such cartridge injection systems are disclosed in U.S. patent application Ser. No. 15/801,281, which was previously incorporated by reference herein.

Accordingly, there exists a need for needle handling devices to removably hold a needle shield apart from but the injection system with a housing but still visually obscuring, concealing, or hiding the needle. In particular, there is a need for needle shield handling devices, which may utilize the existing and relatively well-controlled supply chain of conventionally delivered syringe assemblies.

### SUMMARY

Embodiments are directed to injection systems. In particular, the embodiments are directed to needle shield handling devices and needle obscuring assemblies for injection systems with housings.

In one embodiment, an injection system includes a housing member having a housing body defining a housing interior. The system also includes an injection member disposed in the housing interior. The injection member includes a syringe body having proximal and distal ends and defining a syringe interior. The injection member also includes a stopper member disposed in the syringe interior. The injection member further includes a plunger member coupled to the stopper member and configured to be manipulated to insert the stopper member distally in the syringe interior relative to the syringe body. Moreover, the injection member includes a needle hub assembly coupled to the syringe body at the distal end thereof. The needle hub assembly includes a needle hub and a needle coupled to the needle hub. The housing member includes a needle obscuring member slidably coupled to the housing body. The needle obscuring member is configured to be disposed at least partially over the needle.

In one or more embodiments, the needle obscuring member includes a pair of opposing radially outward extending tabs. The housing body may define a corresponding pair of opposing slots. Each of the pair of opposing radially outward extending tabs may be slidably disposed in respective ones of the corresponding pair of opposing slots. The pair of opposing radially outward extending tabs may be configured to guide axial movement of the needle obscuring member. The pair of opposing radially outward extending tabs may be configured to limit distal and proximal axial movement of the needle obscuring member. The housing body may define an outer knurled surface. The housing body may define a pair of opposing windows configured to facilitate visualization of the syringe body.

In one or more embodiments, the injection member includes a needle shield removably coupled to the needle hub over the needle. The injection system may include a needle shield handling member coupled to the needle shield and configured to facilitate removal of the needle shield from the needle hub to expose the needle. The needle shield handling member may include a pair of flexible tabs configured to interfere with a proximally facing surface of the needle shield to couple the needle shield handling member to the needle shield. The needle obscuring member may define an annular space configured to allow the pair of flexible tabs to flex radially outward over the needle shield to couple the needle shield handling member to the needle shield.

In one or more embodiments, the housing member includes an injection member holder disposed in the housing interior. The injection member may include a compression ring to couple the needle hub assembly to the syringe body. The injection member holder may include a distal slot configured to receive the compression ring to limit axial movement of the injection member in the housing member.

In one or more embodiments, the injection system also includes a plunger manipulation member disposed partially proximal to the housing member and configured to insert the plunger member and the stopper member coupled thereto distally in the syringe interior relative to the syringe body. The plunger manipulation member may be partially disposed in an open proximal end of the housing member.

In another embodiment, an injection system includes a housing member including a housing body defining a housing interior. The system also includes an injection member disposed in the housing interior. The injection member includes a syringe body having proximal and distal ends and defining a syringe interior. The injection member also includes a stopper member disposed in the syringe interior. The injection member further includes a plunger member coupled to the stopper member and configured to be manipulated to insert the stopper member distally in the syringe interior relative to the syringe body. Moreover, the injection member includes a needle hub assembly coupled to the syringe body at the distal end thereof. The needle hub assembly includes a needle hub and a needle coupled to the needle hub. The system further includes a plunger manipulation member having an open distal end and a plunger interface coupled to a proximal end of the plunger member. The housing member includes a needle obscuring member coupled to the housing body. The needle obscuring member is configured to be disposed at least partially over the needle.

In one or more embodiments, the plunger manipulation member defines an outer knurled surface. The housing member may include a ring configured to hold the needle hub and the injection member attached thereto slidably in the housing interior. The ring may be configured to center the injection member in the housing interior.

In one or more embodiments, the housing member defines a proximal end radially outward extending flange. The plunger manipulation member may define a radially inward extending detent configured to interfere with the proximal end radially outward extending flange to limit distal movement of the housing member relative to the plunger manipulation member. The housing member may define a radially outward extending detent distal of the proximal end radially outward extending flange. The radially outward extending detent may be configured to interfere with the radially inward extending detent to limit distal movement of the plunger manipulation member relative to the housing member until an interference between the radially outward extending detent and the radially inward extending detent is overcome. The housing member, the injection member, and the plunger manipulation member may be configured such that applying a distally directed force to the plunger manipulation member while the needle obscuring member is in contact with a patient first moves the injection member and the needle attached thereto into the patient, then moves the plunger member and the stopper member attached thereto distally into the syringe interior to perform an injection.

In still another embodiment, an injection system includes a housing member and an injection member. The housing member includes an outer tube defining a needle obscuring member at a distal end thereof. The housing member also includes a syringe housing disposed at least partially in the outer tube and defining a syringe housing interior. The housing member further includes a handle slidably coupled to the outer tube and the syringe housing. The injection member is disposed in the syringe housing interior. The injection member includes a syringe body having proximal and distal ends and defining a syringe interior. The injection member also includes a stopper member disposed in the syringe interior. The injection member further includes a plunger member coupled to the stopper member and configured to be manipulated to insert the stopper member distally in the syringe interior relative to the syringe body. Moreover, the injection member includes a needle hub assembly coupled to the syringe body at the distal end of the syringe body. The needle hub assembly includes a needle hub, and a needle coupled to the needle hub. The needle obscuring member is configured to be disposed at least partially over the needle.

In one or more embodiments, the syringe housing defines a syringe housing latch adjacent a distal end of the syringe housing. The syringe housing may define a syringe housing rib adjacent a proximal end of the syringe housing. The syringe housing latch and the syringe housing rib may be configured to limit longitudinal movement of the injection member relative to the syringe housing. The injection system may include a shroud coupled to the distal end of the outer tube. The shroud defines a shroud arm at a proximal end of the shroud, a shroud slot at a distal end of the shroud configured to align with a tab defined by the outer tube at a distal end of the outer tube, and a shroud opening at the distal end of the shroud.

In one or more embodiments, the injection member includes a needle shield removably coupled to the needle hub and covering the needle. The needle shield may define a needle shield distal opening at a distal end of the needle shield, and a needle shield longitudinal slot along a radially outward surface of the needle shield. The injection member may include a needle shield cap. The needle shield cap may define a needle shield cap latch configured to cooperate with the needle shield distal opening to couple the needle shield cap to the needle shield, a needle shield cap guide column configured to cooperate with the needle shield longitudinal slot to couple the needle shield cap to the needle shield, and a needle shield cap detent configured to cooperate with the shroud opening to couple the needle shield cap to the shroud. When the needle shield cap and the needle shield are removably coupled to the needle hub, the needle shield cap interferes with the shroud arm to maintain a radial position of the shroud arm relative to the distal end of the syringe housing, thereby preventing the syringe housing and the injection member contained in the syringe housing from moving distally relative to the shroud and the outer tube until the needle shield cap and the needle shield are removed from the needle hub.

In one or more embodiments, the injection system includes a needle insertion spring disposed around the syringe housing and configured to provide a needle insertion resistance to distal movement of the syringe housing relative to the outer tube. The needle insertion resistance may be substantially equal to a stopper advancement resistance to distal advancement of the stopper member relative to the syringe body.

In one or more embodiments, the injection system includes a rotating cuff disposed around a proximal portion of the syringe housing and translationally coupled to the syringe housing. The rotating cuff may define a radially extending block at a proximal end of the rotating cuff, and an angled block at a distal end of the rotating cuff. The handle may define a distally facing wall configured to cooperate with the radially extending block to prevent distal movement of the handle relative to the rotating cuff and the syringe housing coupled thereto before the syringe housing and the rotating cuff coupled thereto move distally relative to the outer tube to prevent premature ejection of a liquid from the syringe interior. The outer tube may define an angled slot at a proximal end of the outer tube configured to cooperate with the angled block to control rotation of the rotating cuff relative to the outer tube and the handle. Moving the syringe housing and the rotating cuff coupled thereto distally relative to the outer tube may rotate the rotating cuff relative to the syringe housing, the outer tube and the handle. Rotating the rotating cuff relative to the syringe housing, the outer tube and the handle may rotate the radially extending block away from the distally facing wall and into a slot defined on an inner surface of the handle to allow the handle to move distally relative to the rotating cuff and the syringe housing coupled thereto to eject a liquid from the syringe interior.

In one or more embodiments, the syringe housing may define a syringe housing anti-return latch. The outer tube may define an anti-return window configured to cooperate with the syringe housing anti-return latch to prevent the syringe housing from moving proximally relative to the outer tube after the syringe housing moves distally relative to the outer tube to a distal limit of travel of the syringe housing.

In one or more embodiments, the syringe housing defines a syringe housing radially outward extending tab configured to cooperate with a syringe housing tab slot defined in an inner surface of the handle to prevent the handle from rotating in a first direction or moving proximally relative to the syringe housing while allowing the handle to move distally relative to the syringe housing after rotation of the rotating cuff relative to the handle. The outer tube may define an outer tube radially outward extending tab configured to cooperate with an outer tube tab slot defined in an inner surface of the handle to prevent the handle from moving proximally relative to the outer tube while allowing the handle to move distally relative to the outer tube. The syringe housing may define a syringe housing external housing latch configured to cooperate with a syringe housing latch slot defined in an inner surface of the handle to prevent the handle from rotating in a second direction relative to the syringe housing after assembly.

In one or more embodiments, the injection system includes a handle cap disposed at a proximal end of the handle. The handle cap may define a handle cap latch configured to cooperate with a proximal end wall defined on an inner surface of the handle to prevent the handle cap from moving proximally relative to the handle. The handle cap may define a plunger member arm configured to cooperate with a distally facing surface at a proximal end of the plunger member to prevent the handle from moving proximally relative to the plunger member.

The aforementioned and other embodiments of the invention are described in the Detailed Description which follows.

### BRIEF DESCRIPTION OF THE DRAWINGS

FIGS. 1A to 5C illustrate various aspects of conventional injection syringe configurations.

FIGS. 6 to 9 are perspective, front, side, and longitudinal cross-sectional views of an injection system having a housing member, a needle obscuring member, and a needle shield handling member in a transport/storage configuration according to some embodiments.

FIG. 10 is a partially exploded view of the injection system depicted in FIGS. 6 to 9.

FIG. 11 is a perspective view of the injection system depicted in FIGS. 6 to 10 with the needle shield handling member and the needle shield contained therein separated from the other components of the injection system.

FIGS. 12 and 13 are perspective and longitudinal cross-sectional views of the injection system depicted in FIGS. 6 to 11 in a ready-to-use configuration.

FIGS. 14 and 15 are perspective and longitudinal cross-sectional views of the injection system depicted in FIGS. 6 to 13 in an extended configuration.

FIG. 16 is a longitudinal cross-sectional view of the injection system depicted in FIGS. 6 to 15 in an injection-complete configuration.

FIG. 17 is a longitudinal cross-sectional view of the injection system depicted in FIGS. 6 to 16 in a needle-retracted/safe configuration.

FIG. 18 is a perspective view of a needle obscuring member configured for use with the injection system depicted in FIGS. 6 to 17, according to some embodiments.

FIG. 19 is a perspective view of a front portion of a housing body for use with the injection system depicted in FIGS. 6 to 17, according to some embodiments.

FIGS. 20 and 21 are partially cutaway front views of the injection system depicted in FIGS. 6 to 17 in transport (FIG. 20) and ready-to-use (FIG. 21) configurations with the front portion of the housing body omitted for clarity.

FIGS. 22A and 22B are perspective views of the injection system depicted in FIGS. 6 to 17 in ready-to-use (FIG. 22A) and extended (FIG. 22B) configurations with the front portion of the housing body omitted for clarity.

FIG. 23 is a partially cutaway perspective view of a needle shield handling member and a needle shield contained therein for use with the injection system depicted in FIGS. 6 to 17, according to some embodiments.

FIG. 24 is a partially cutaway perspective view of the injection system depicted in FIGS. 6 to 17 with the needle shield handling member and the needle shield contained therein removably attached to the other components of the injection system.

FIGS. 25A to 25D are partially cutaway front views of the injection system depicted in FIGS. 6 to 17 with the front portion of the housing body omitted for clarity.

FIGS. 26 to 28 are perspective, front, and longitudinal cross-sectional views of an injection system having a housing member, a needle obscuring member, and a needle shield handling member in a transport/storage configuration according to some embodiments.

FIG. 29 is a partially exploded view of the injection system depicted in FIGS. 26 to 28.

FIG. 30 is a perspective view of the injection system depicted in FIGS. 26 to 29 with the needle shield handling member and the needle shield contained therein separated from the other components of the injection system.

FIG. 31 is a perspective view of the injection system depicted in FIGS. 26 to 30 in a ready-to-use configuration.

FIG. 32 is a longitudinal cross-sectional view of the injection system depicted in FIGS. 26 to 31 in an extended configuration.

FIGS. 33 and 34 are perspective and longitudinal cross-sectional views of the injection system depicted in FIGS. 26 to 32 in an injection-complete configuration.

FIG. 35 is a longitudinal cross-sectional view of the injection system depicted in FIGS. 26 to 34 in a needle-retracted/safe configuration.

FIG. 36 is a perspective view of a housing member configured for use with the injection system depicted in FIGS. 26 to 35, according to some embodiments.

FIG. 37 is a partially cutaway front view of a ring disposed around an injection member for use with the injection system depicted in FIGS. 26 to 35, according to some embodiments.

FIG. 38 is a longitudinal cross-sectional view of a housing body for use with the injection system depicted in FIGS. 26 to 35, according to some embodiments.

FIGS. 39 and 40 are detailed partially cutaway perspective views of the interface between the plunger manipulation member and the housing member of the injection system depicted in FIGS. 26 to 35 in transport or ready-to-use configurations.

FIGS. 41 and 42 are perspective and partially cutaway perspective views of a needle shield handling member and a needle shield contained therein for use with the injection system depicted in FIGS. 26 to 35, according to some embodiments.

FIG. 43 is a longitudinal cross-sectional view of a needle shield handling member coupled to the injection system depicted in FIGS. 26 to 35 with the needle shield omitted for clarity.

FIG. 44 is a partial cutaway perspective view of a needle shield handling member coupled to the injection system depicted in FIGS. 26 to 35.

FIGS. 45A to 45E are front views of the injection system depicted in FIGS. 26 to 35.

FIG. 46 is a partially exploded view of an injection system, according to some embodiments.

FIG. 47 is a perspective view of a handle configured for use with the injection system depicted in FIG. 46, according to some embodiments.

FIGS. 48A and 48B are perspective views of a handle cap configured for use with the injection system depicted in FIG. 46, according to some embodiments.

FIGS. 49A and 49B are detailed longitudinal cross-sectional views of the proximal end of the injection system depicted in FIG. 46, according to some embodiments.

FIGS. 50A and 50B are perspective views of an outer tube configured for use with the injection system depicted in FIG. 46, according to some embodiments.

FIG. 51 is a perspective view of a shroud configured for use with the injection system depicted in FIG. 46, according to some embodiments.

FIGS. 52A and 52B are detailed and more detailed longitudinal cross-sectional views of the distal end of the injection system depicted in FIG. 46, according to some embodiments.

FIGS. 53 and 54 are perspective and partial cutaway perspective views of a needle shield cap configured for use with the injection system depicted in FIG. 46, according to some embodiments.

FIG. 55 is a detailed longitudinal cross-sectional view of the distal end of the injection system depicted in FIG. 46, according to some embodiments.

FIGS. 56 and 57 are perspective views of a syringe housing configured for use with the injection system depicted in FIG. 46, according to some embodiments.

FIG. 58 is a perspective view of a rotating cuff configured for use with the injection system depicted in FIG. 46, according to some embodiments.

FIGS. 59A to 60B are detailed longitudinal cross-sectional views of the injection system depicted in FIG. 46, according to some embodiments.

FIGS. 61A and 61B are side and detailed longitudinal cross-sectional views of the injection system depicted in FIG. 46 in a ready-to-use configuration, according to some embodiments. The handle is omitted from FIG. 61A for clarity.

FIGS. 62A and 62B are side and detailed longitudinal cross-sectional views of the injection system depicted in FIG. 46 in an extended configuration, according to some embodiments. The handle is omitted from FIG. 62A for clarity.

FIGS. 63A and 63B are detailed and more detailed partially cutaway perspective views of the injection system depicted in FIG. 46, according to some embodiments.

FIG. 64 is a detailed longitudinal cross-sectional view of the injection system at the proximal end of the syringe housing depicted in FIG. 46, according to some embodiments.

FIGS. 65A, 65B, and 65C are axial cross-sectional (FIGS. 65A and 65C) and detailed cutaway perspective (FIG. 65B) views of the injection system depicted in FIG. 46, according to some embodiments.

FIGS. 66 to 68 are perspective, partial cutaway perspective, and longitudinal cross-sectional views of an injection system having a housing member, a syringe housing, an injection member, and a needle shield handling member in a transport/storage configuration according to some embodiments.

FIGS. 69 to 71 are perspective, partial cutaway perspective, and longitudinal cross-sectional views of an injection system having a housing member, a syringe housing, and an injection member in a ready-to-use configuration according to some embodiments.

FIGS. 72 to 74 are perspective, partial cutaway perspective, and longitudinal cross-sectional views of an injection system having a housing member, a syringe housing, and an injection member in an extended configuration according to some embodiments.

FIGS. 75 to 77 are perspective, partial cutaway perspective, and longitudinal cross-sectional views of an injection system having a housing member, a syringe housing, and an injection member in an injection-complete configuration according to some embodiments.

FIGS. 78 to 80 are perspective, partial cutaway perspective, and longitudinal cross-sectional views of an injection system having a housing member, a syringe housing, and an injection member in a needle-retracted/safe configuration according to some embodiments.

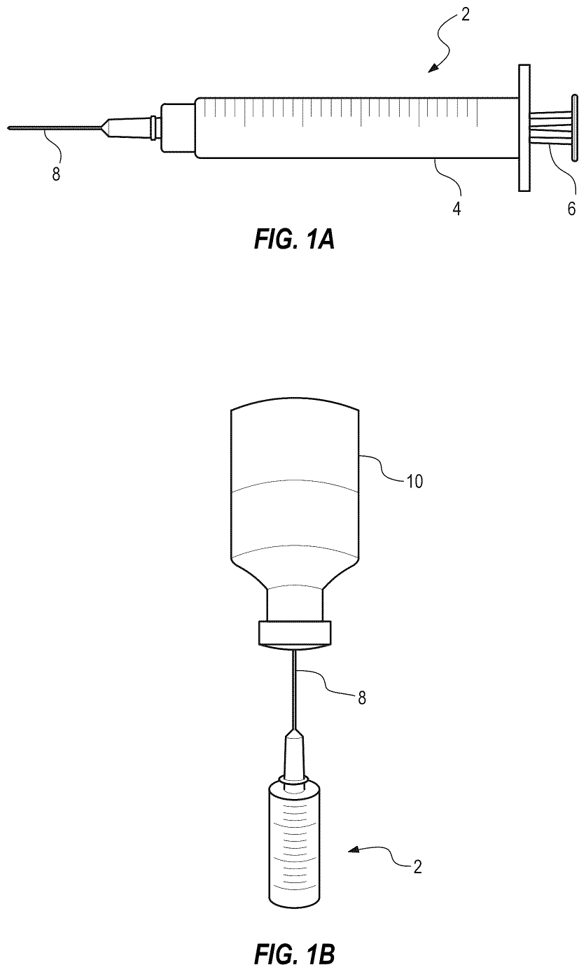

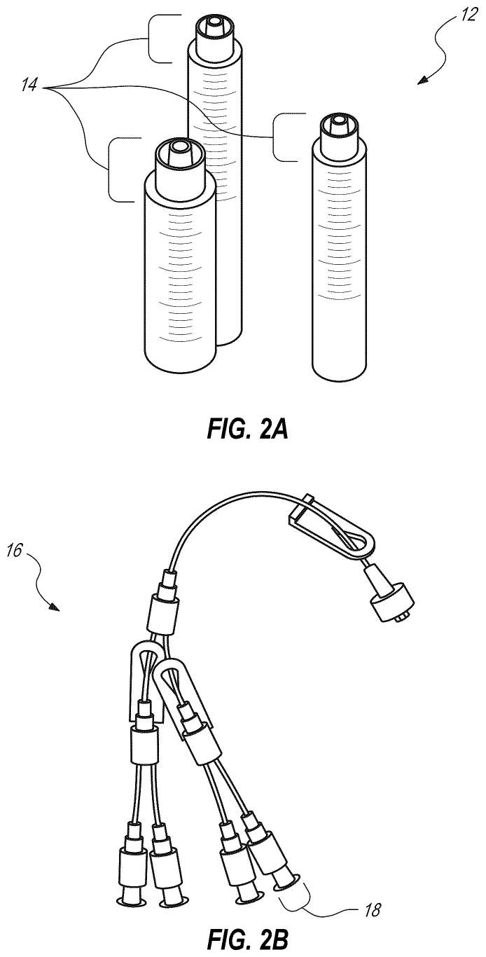

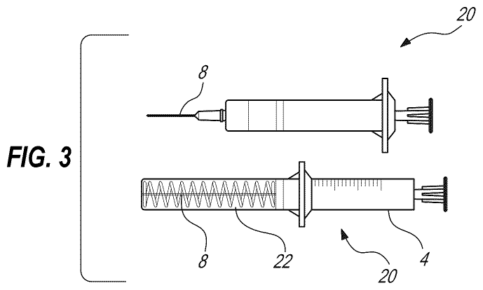

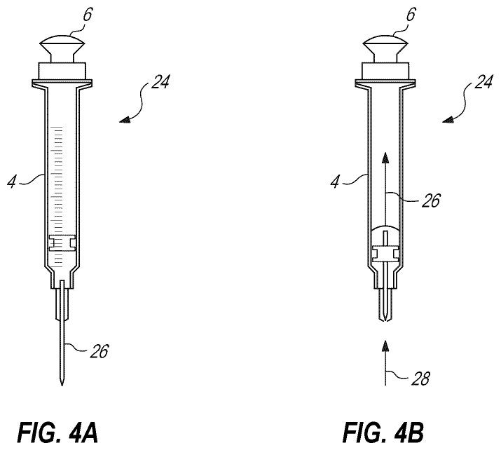

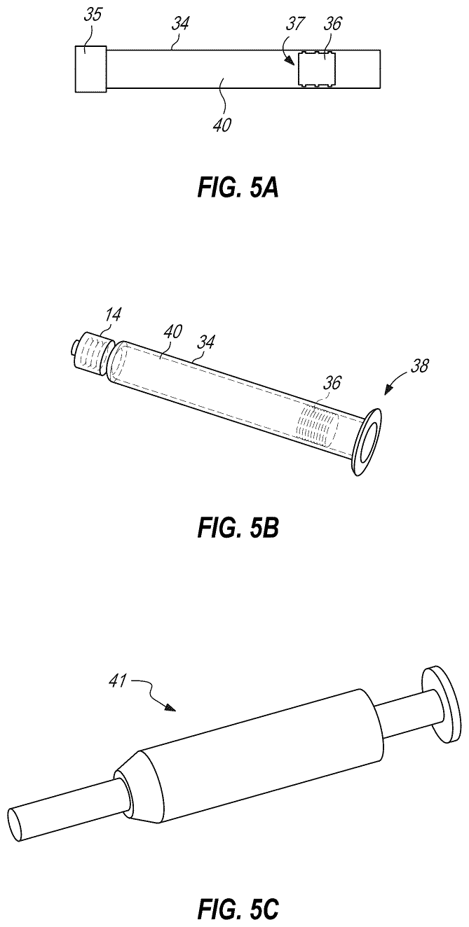

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

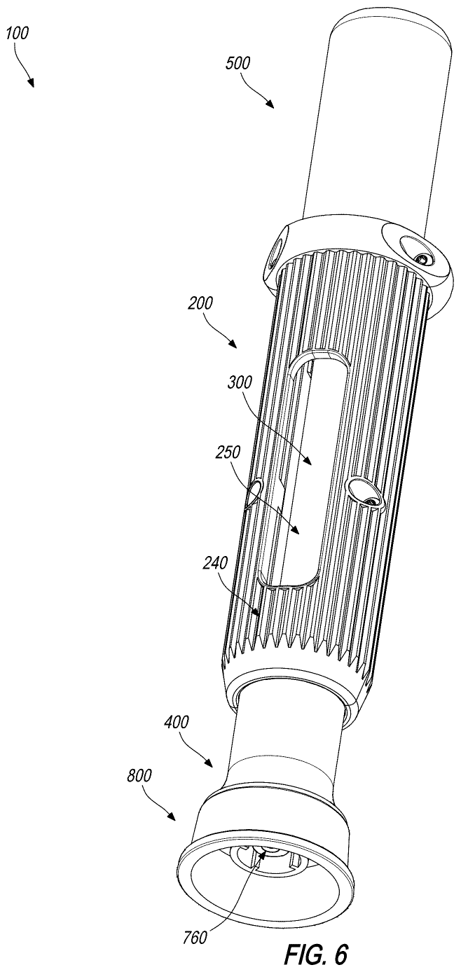

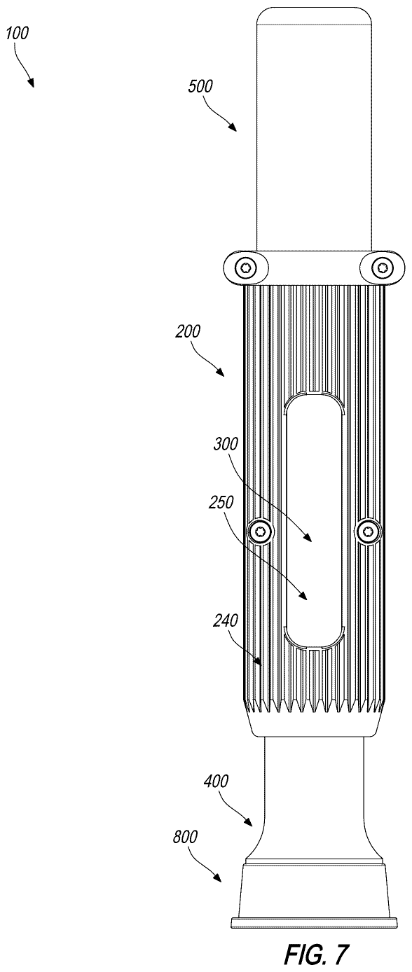

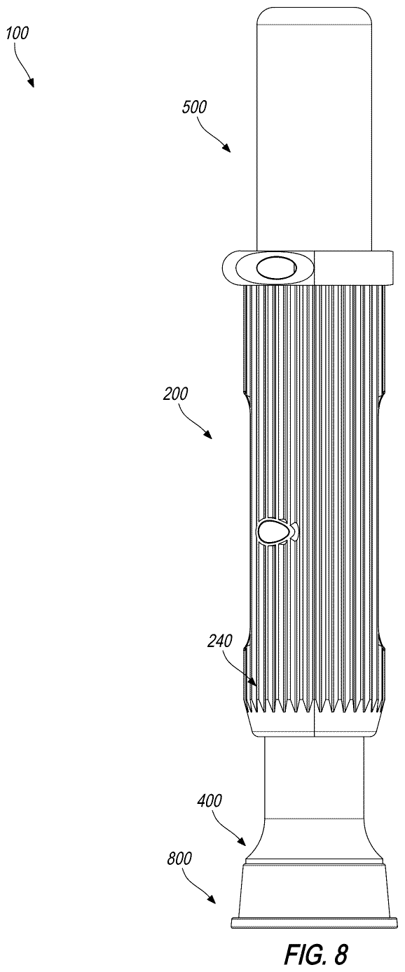

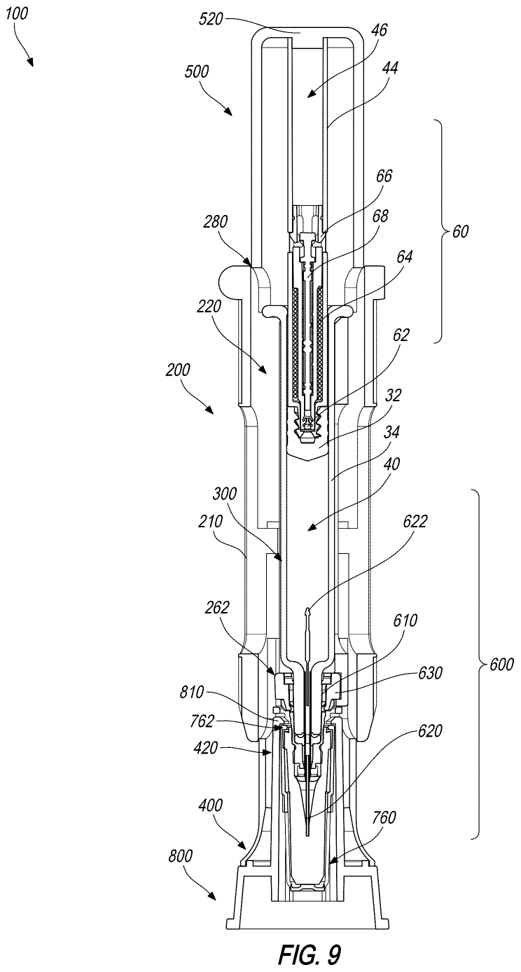

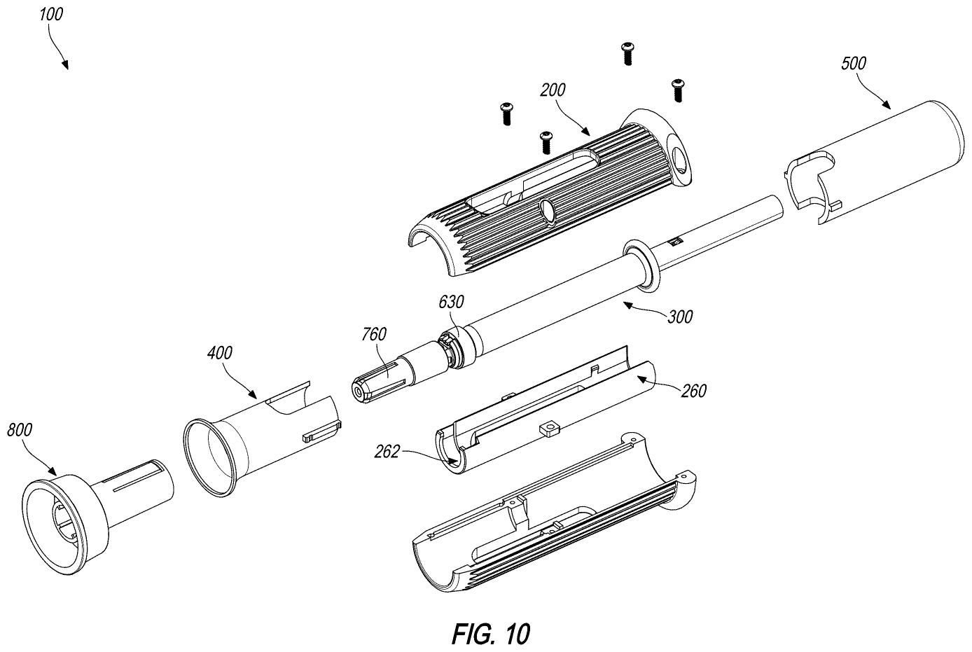

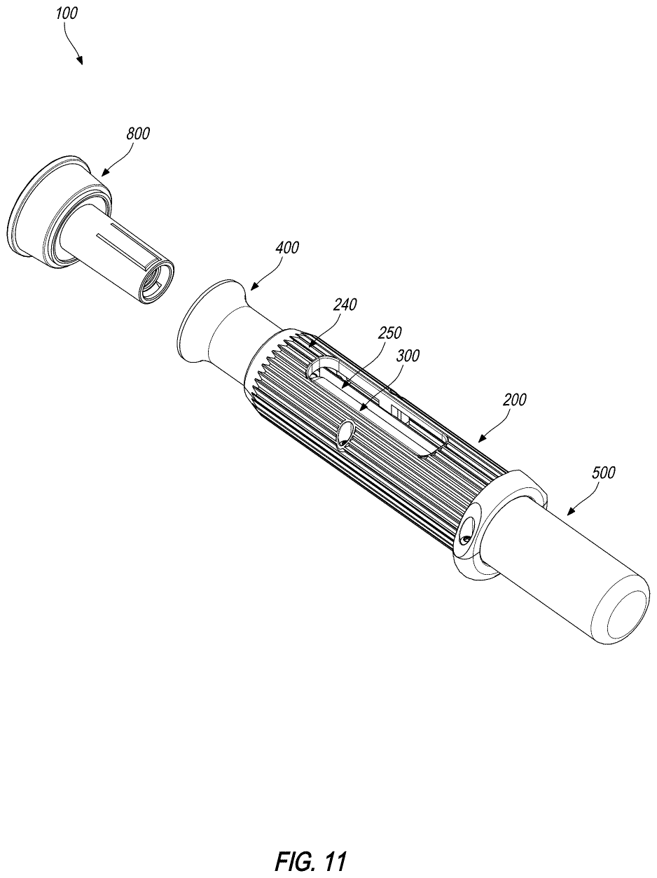

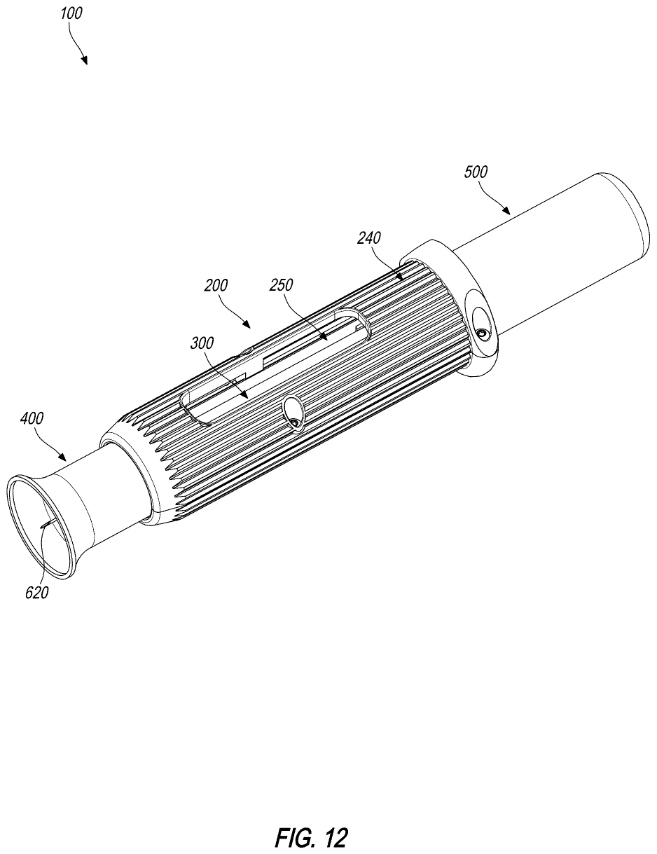

FIGS. 6 to 8 depict an injection system (100) having a housing member (200), a needle obscuring member (400), and a needle shield handling member (800), according to some embodiments. The injection system (100) also includes an injection member (300) and a plunger manipulation member (500) both at least partially disposed in the housing member (200).
FIGS. 9 and 10 are longitudinal cross-sectional and exploded views of the injection system (100). The housing member (200) includes a housing body (210) defining a housing interior (220). The injection member (300) is at least partially disposed in the housing interior (220). Disposing the injection member (300) in the housing member (200) gives the injection system (100) an injection pen form factor. As shown in FIGS. 11 and 12, the housing body (210) defines a pair of opposing windows configured to facilitate visualization of the injection member (100) housed in the housing member (200). The housing body (210) also defines a knurled external surface (240) to facilitate handling of the housing member (200) by the user.

The injection member (300) includes a conventional off-the-shelf pre-filled syringe body (34) defining a medicine reservoir or syringe interior (40). The medicine reservoir or syringe interior (40) is in fluid communication with a syringe needle (620), as shown in FIG. 9. While the injection member (300) is a single chamber injection system, the injection system (100) can be utilized with multiple chamber injection systems, such as those described in U.S. patent application Ser. No. 18/300,125, which was previously incorporated by reference herein.

As shown in FIG. 9, the injection member (300) includes a needle hub assembly (600) disposed at the distal end of the syringe body (34). The needle hub assembly (600) includes a needle hub (610) and the needle (620), where the needle (620) is coupled to the needle hub (610). The needle hub assembly (600) includes a compression ring (630). The needle (620) is covered by a needle shield (760) for storage and before use of the injection system (100).

As shown in FIG. 9, the injection member (300) also includes a plunger member (44) inserted into the proximal end of the syringe body (34). The injection member (300) further includes a stopper member (32) coupled to the plunger member (44). The plunger member (44) is configured to be manipulated to insert the stopper member (32) distally in the syringe interior (40) relative to the syringe body (34) to deliver liquid medicine disposed within the syringe interior (40) into the patient via the needle (620). The stopper member (32) may comprise a standard butyl rubber and may be coated, such as with a biocompatible lubricious coating (e.g., PTFE), to facilitate sealing and relative motion characteristics against the associated syringe body structure and material. Medicine delivery to a patient by the injection member (300) is controlled by distal insertion of the plunger member (44) relative to the syringe body (34) to various degrees by a user after the needle (620) is inserted into a patient.

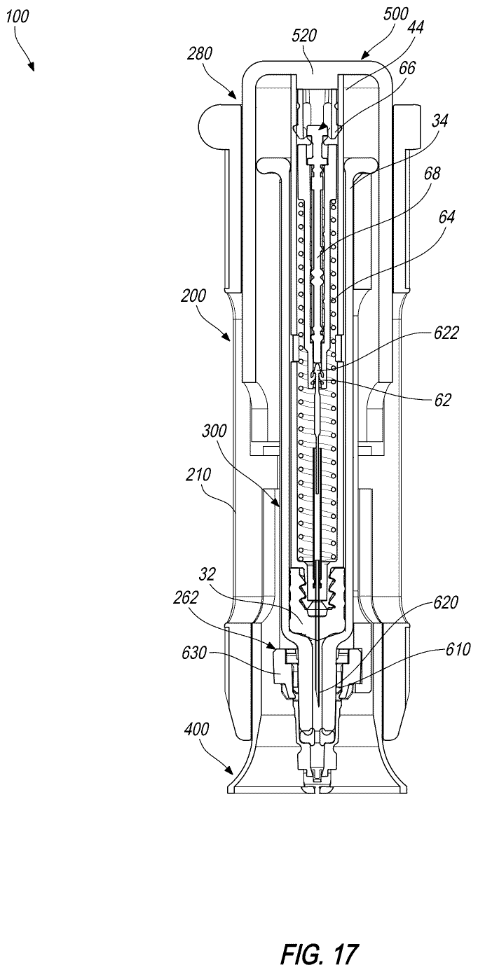

The plunger member (44) defines a plunger interior (46) in which a needle retraction system (60) is substantially disposed. The needle retraction system (60) includes a needle coupling member (62), an energy storage member/spring (64), a spring latch (66), and a latch actuation member (68). The needle coupling member (62) is configured to capture a proximal end member (622) of the needle (620) to operatively couple the needle (620) to the energy storage member/spring (64). The spring latch (66) is configured to temporarily hold the energy storage member/spring (64) in a compressed/energized state as shown in FIG. 9. The latch actuation member (68) is configured to be moved by the proximal end member (622) of the needle (620) to release the energy storage member/spring (64) from the compressed/energized state to transform to an expanded/deenergized state to which the energy storage member/spring (64) is biased (see e.g., FIG. 17). Further details regarding the needle retraction system (60) are described in U.S. patent application Ser. No. 14/696,342, which was previously incorporated by reference herein.

As shown in FIGS. 9 and 10, the injection system (100) also includes a plunger manipulation member (500) inserted into an open proximal end (280) of the housing member (200). The plunger manipulation member (500) defines a plunger interface (520) configured to couple to a proximal end of the plunger member (44), which does not have a thumbpad.

The injection system (100) further includes an injection member holder (260) disposed in the housing interior (220) and configured to center the injection member (300) in the housing member (200). The injection member holder (260) defines a distal slot (262) configured to hold the compression ring (630) of the needle hub assembly (600) to fix the injection member (300) to the housing member (200).

The injection system (100) also includes a needle obscuring member (400) slidably coupled to a distal end of the housing member (200). The needle obscuring member (400) is also shown in FIG. 18 and includes an outwardly flaring distal end configured to obscure a distal end of the needle (620) from a patient during use. The needle obscuring member (400) also includes a pair of opposing radially outward extending tabs (410). The pair of opposing radially outward extending tabs (410) are configured to cooperate with a corresponding pair of opposing slots (230) defined by the housing body (210) as shown in FIG. 19. The pair of opposing radially outward extending tabs (410) and the corresponding pair of opposing slots (230) are configured to slidably couple the needle obscuring member (400) to the housing member (200). U.S. patent application Ser. No. 18/300,125 and U.S. Provisional Patent Application Ser. No. 63/596,404, which were previously incorporated by reference herein, describe injection system components that have functions (e.g., needle obscuring) similar to those of the needle obscuring member (400) described herein. The pair of radially outward extending tabs (410) may define detent notches (412), which are configured to line up with corresponding bumps (212) on the housing member (200) to locate the needle obscuring member in a distally extended position and/or a retracted position. The detent notches (412) for the distally extended position of the needle obscuring member (400) may be overcome by the patient when the distal end of the needle obscuring member (400) is on the patient's skin and the patient applies a distally directed injection force to the plunger manipulation member (500) to insert the needle (620) into the patient.

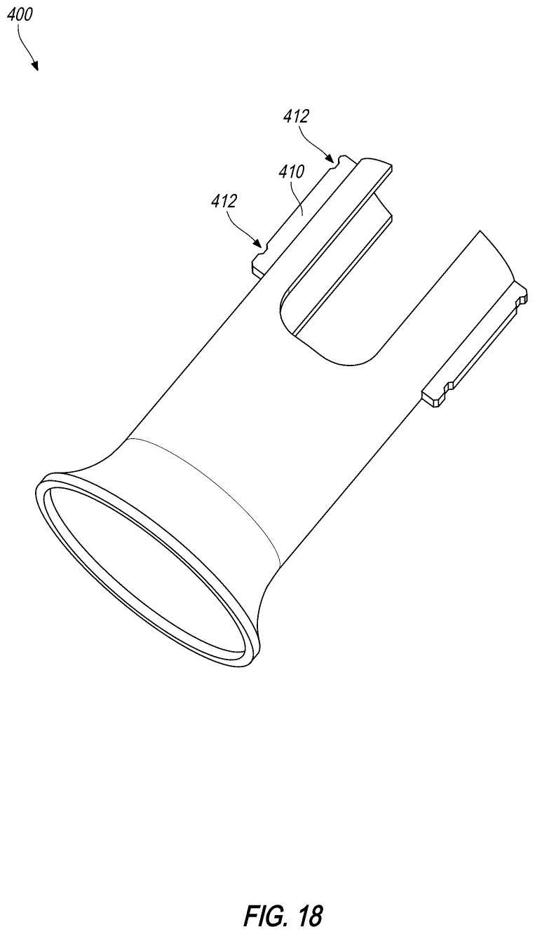

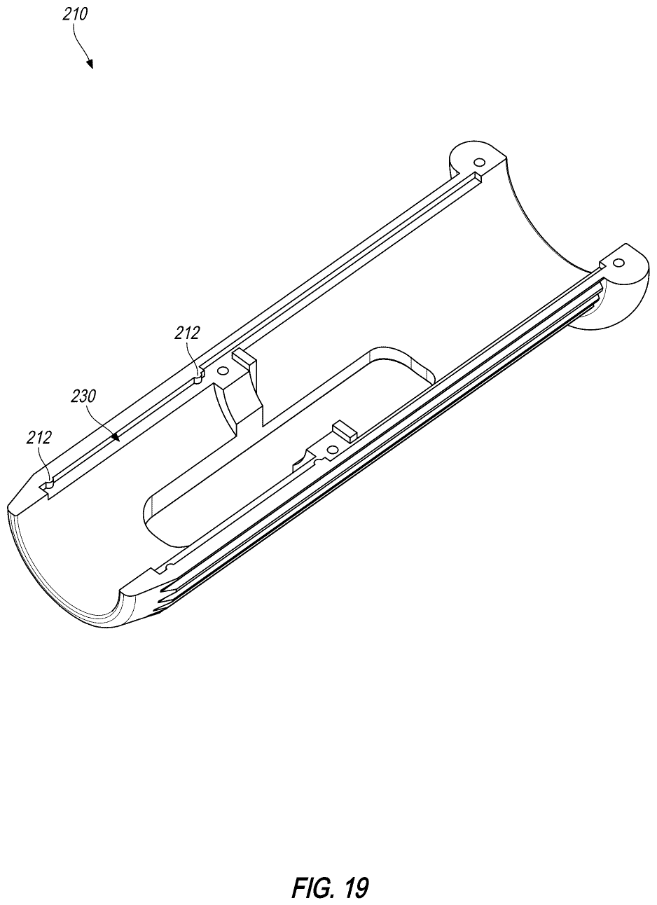

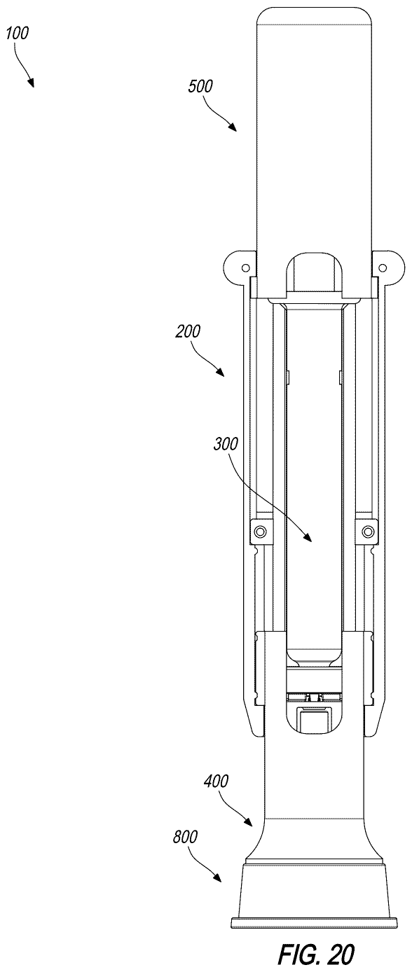

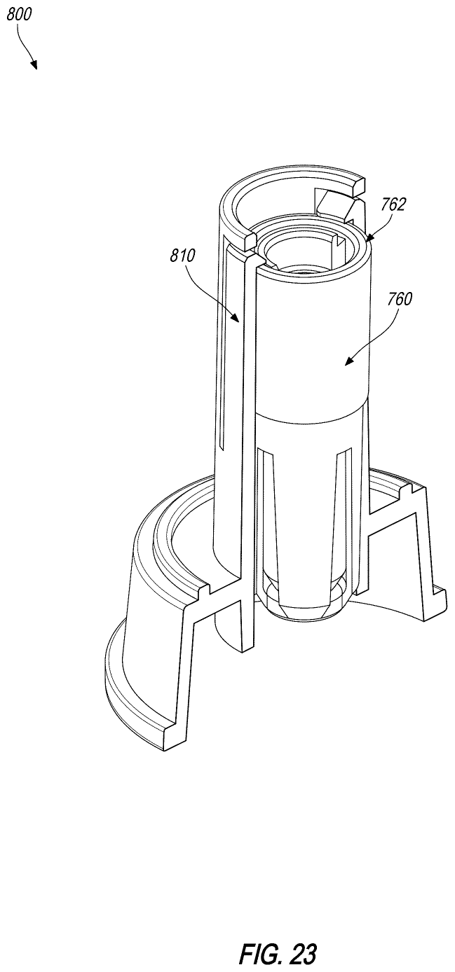

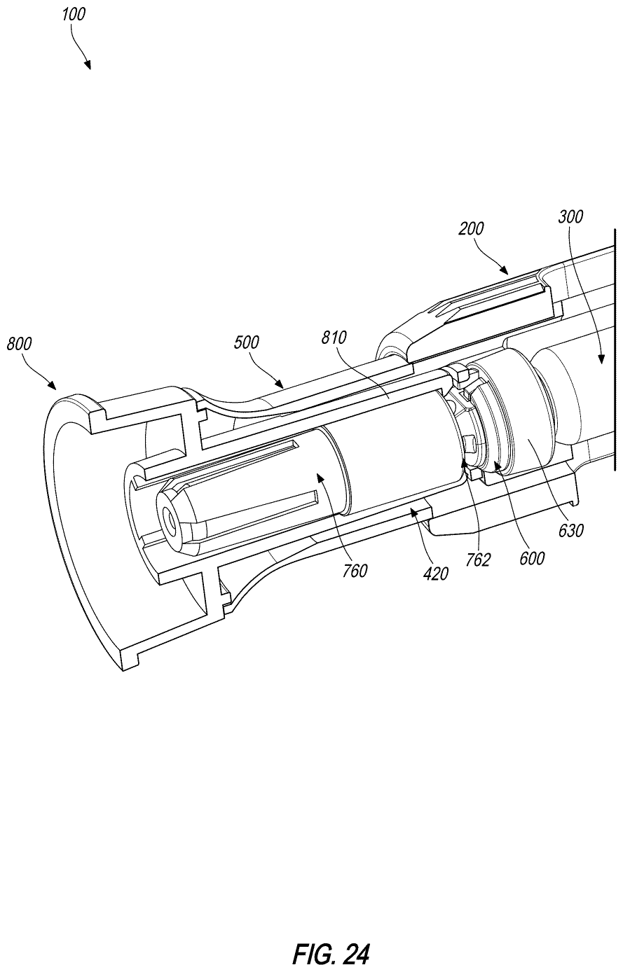

The injection system (100) further includes a needle shield handling member (800), which is also shown in FIGS. 23 and 24. The needle shield handling member (800) is configured to allow a user to remove a needle shield (760) from the needle hub assembly (600) of an injection member (300) disposed inside of a housing member (200) and a needle obscuring member (400) such as in the transport/storage configuration depicted in FIGS. 6 to 9 and 20. The needle shield handling member (800) defines a pair of flexible tabs (810) configured to interfere with a proximally facing surface (762) of the needle shield (760) to couple the needle shield handling member (800) to the needle shield (760). Coupling the needle shield handling member (800) to the needle shield (760) allows a user to pull the needle shield handling member (800) distally to remove the needle shield (760) from the needle hub assembly (600) as shown in FIG. 11.

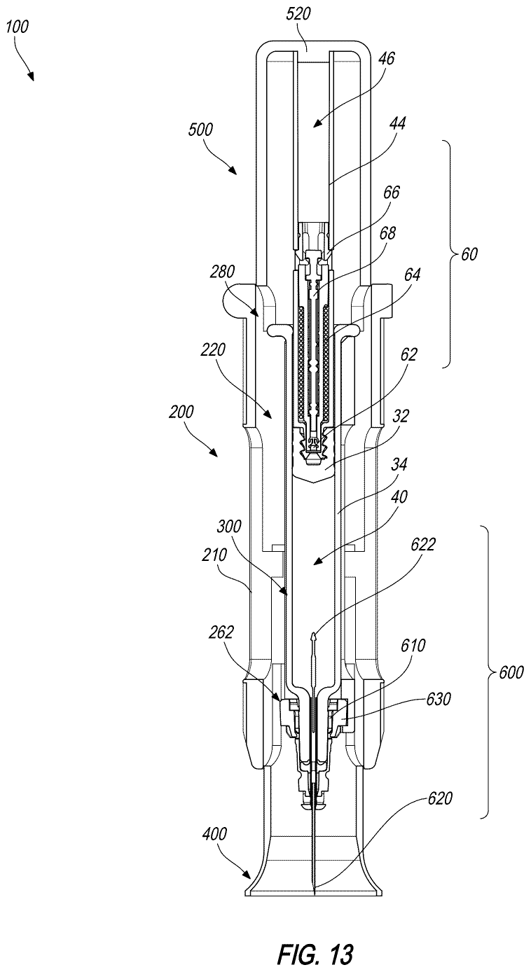

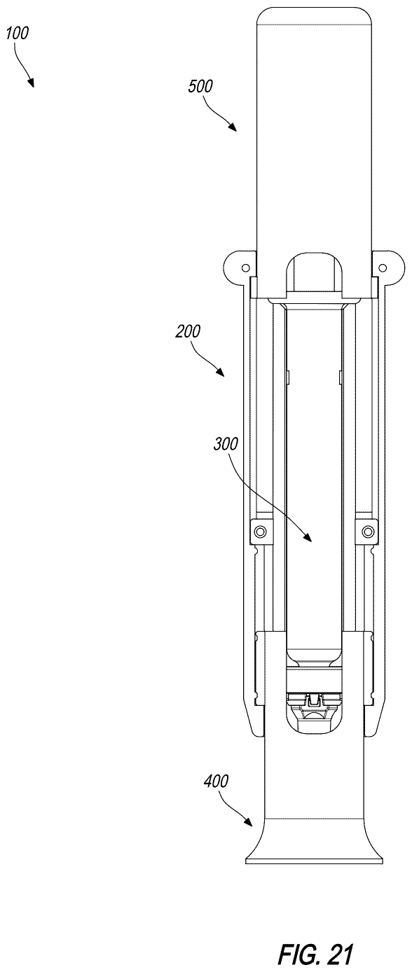

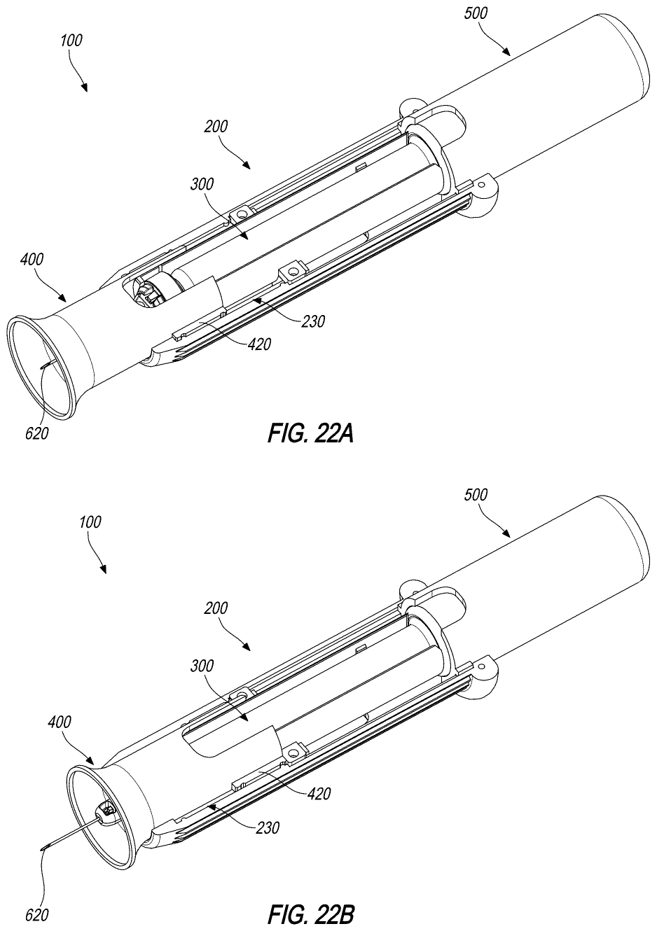

After removing the needle shield handling member (800) and the needle shield (760) attached thereto, the injection system (100) depicted in FIGS. 11 to 13, 21, 22A, and 25A is in a ready-to-use configuration. In the ready-to-use configuration, the needle obscuring member (400) visually hides the sharp distal end of the needle (620) from the patient. The needle obscuring member (400) may also tactilely hide the sharp distal end of the needle (620) from the patients by touching the skin of the patient at substantially the same time that the sharp distal end of the needle (620) touches the skin of the patient. In a first step in an injection method, the injection system (100) in the ready-to-use configuration is disposed in contact with a patient such that the needle obscuring member (500) and the sharp distal end of the needle (620) is in contact with the skin of the patient. In an alternative embodiment, the needle obscuring member (400) may be configured to touch the skin before the needle (620) touches the patient's skin, preventing piercing of the patient's skin until the injection is initiated.

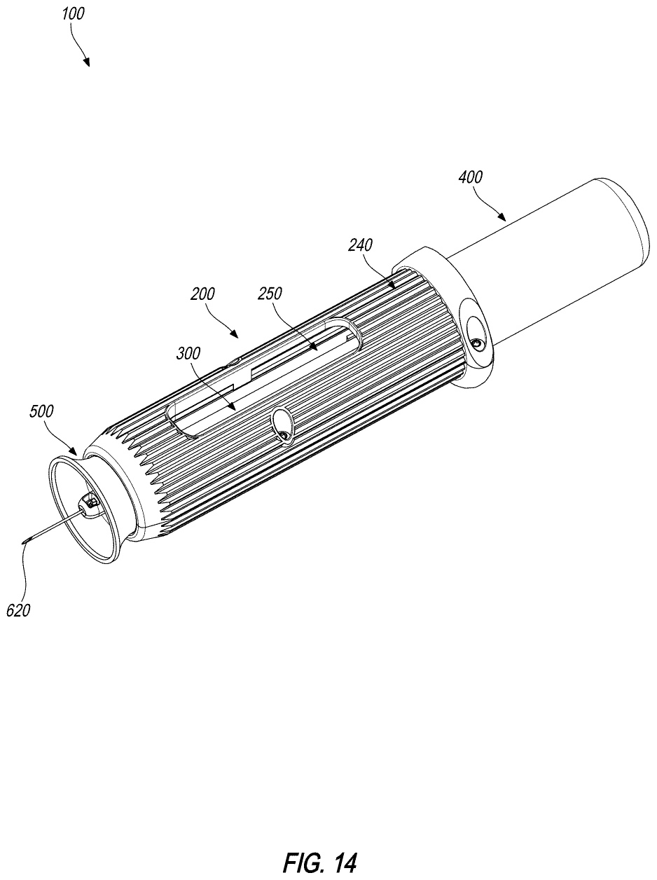

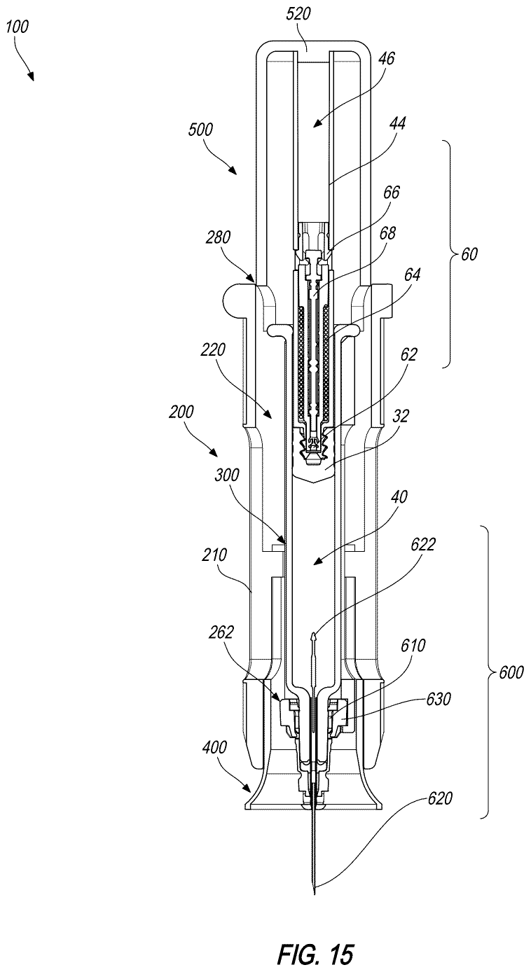

In the next step in the injection method, with the needle obscuring member (400) of the injection system (100) in contact with the skin of the patient, distally directed force is applied to the housing member (200), which is grasped in a hand of a user to insert the needle (620) into the patient and to move the needle obscuring member (400) slidably into the housing member (200). Movement of the needle obscuring member (400) proximally into the housing member (200) is limited by interference between the pair of opposing radially outward extending tabs (410) and the corresponding pair of opposing slots (230), thereby limiting the depth a needle (620) will be inserted into a patient. Inserting the needle (620) into the patient and moving the needle obscuring member (400) proximally into the housing member (200) transforms the injection system (100) from the ready-to-use configuration depicted in FIGS. 11 to 13, 21, 22A, and 25A to the extended configuration depicted in FIGS. 14, 15, 22B, and 25B.

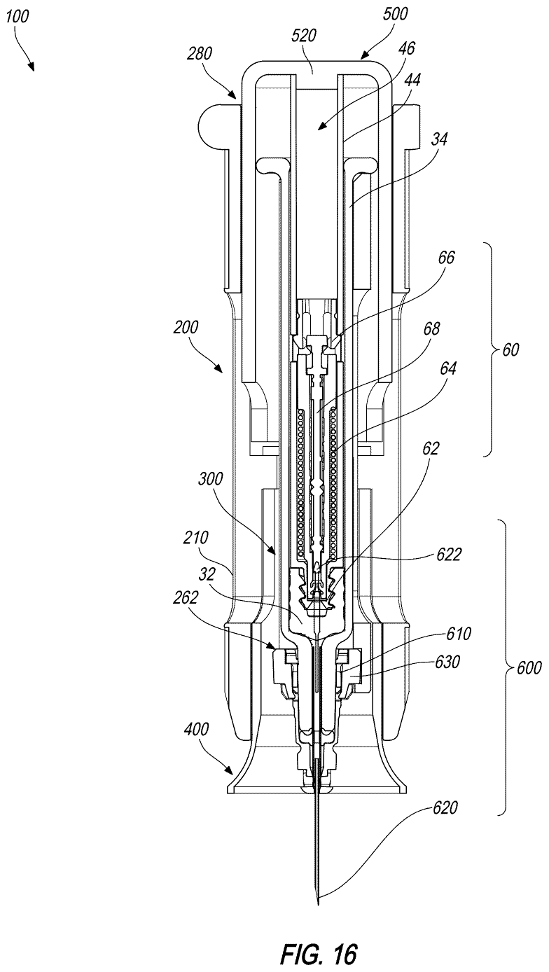

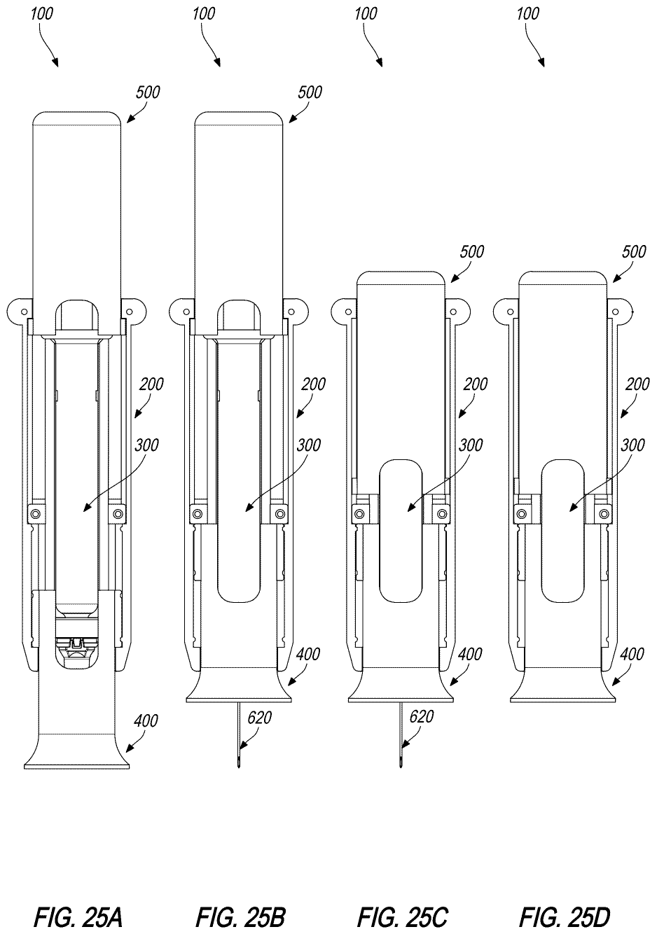

In the next step in the injection method, with the needle (620) inserted into a patient, distally directed force is applied to a proximal end of the plunger manipulation member (500) to move the plunger manipulation member (500), the plunger member (44) attached thereto, and the stopper member (32) attached to the plunger member (44) distally. Moving the stopper member (32) distally in the syringe interior (40) ejects a liquid medicine from the syringe interior (40) through the needle (620) and into the patients. Applying sufficient distally directed force to the plunger manipulation member (500) to move the stopper member (32) to a distal end of the syringe body (34) completes the injection and converts the injection system (100) from the extended configuration depicted in FIGS. 14, 15, 22B, and 25B to the injection-complete configuration depicted in FIGS. 16 and 25C.

From the injection-complete configuration depicted in FIGS. 16 and 25C, continued application of distally directed force to the plunger manipulation member (500) the proximal end member (622) of the needle (620) is both captured by/coupled to the needle coupling member (62) and moves the latch actuation member (68) to release the spring latch (66) thereby allowing the energy storage member/spring (64) to transform from the compressed/energized state to transform to an expanded/deenergized state. When transforming from the compressed state to the expanded state, the energy storage member/spring (64) pulls the needle coupling member (62) and the needle (620) attached thereto proximally into the injection system (100) until the sharp distal end of the needle (620) is disposed in the needle hub (610) and a distal end of the syringe body (34), thereby rendering the injection system (100) safe for disposal. Retracting the needle after injection transforms the injection system (100) from the injection-complete configuration depicted in FIGS. 16 and 25C to the needle-retracted/safe configuration depicted in FIGS. 17 and 25D.

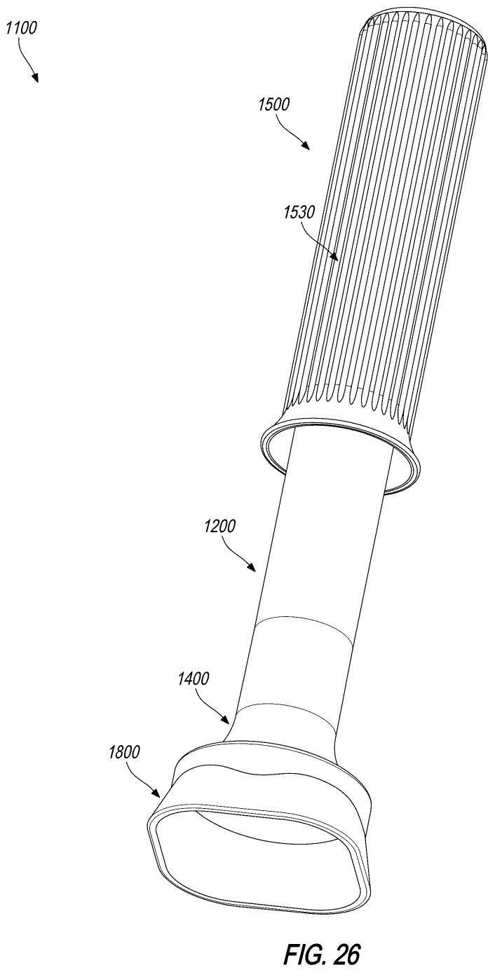

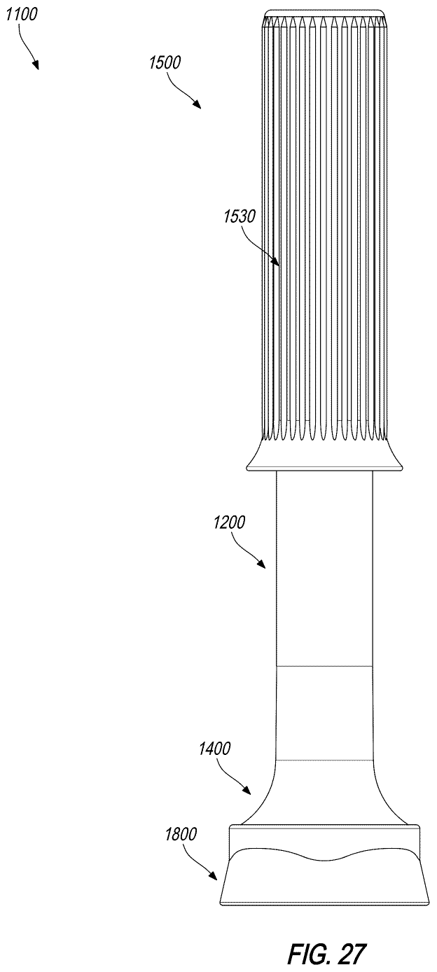

FIGS. 26 and 27 depict an injection system (1100) having a housing member (1200) and a needle shield handling member (1800), according to other embodiments. The injection system (1100) also includes an injection member (300) at least partially disposed in the housing member (1200), and a plunger manipulation member (1500) disposed partially around a proximal end of the housing member (1200). The housing member (1200) includes a needle obscuring member (1400) at a distal end thereof. The needle obscuring member (1400) is a flared portion at a distal end of the housing member (1200).

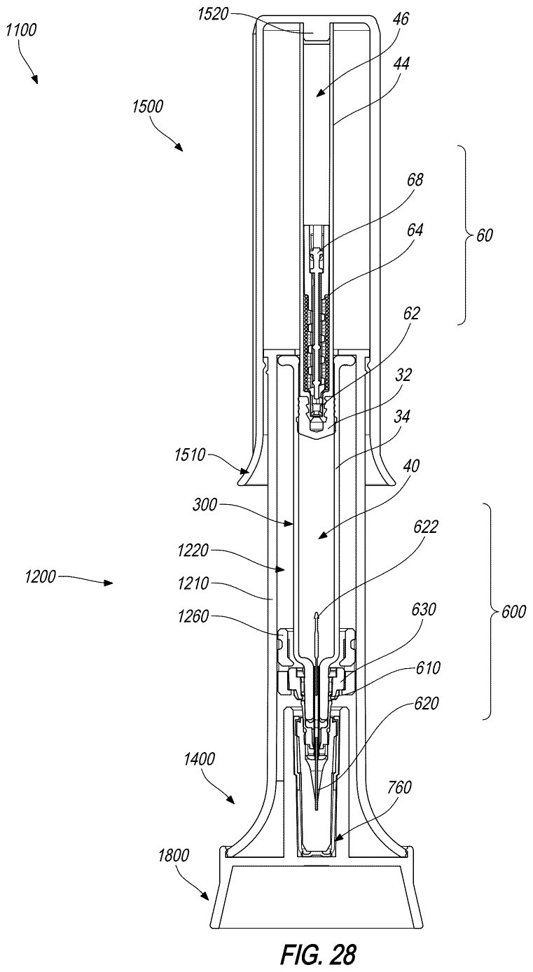

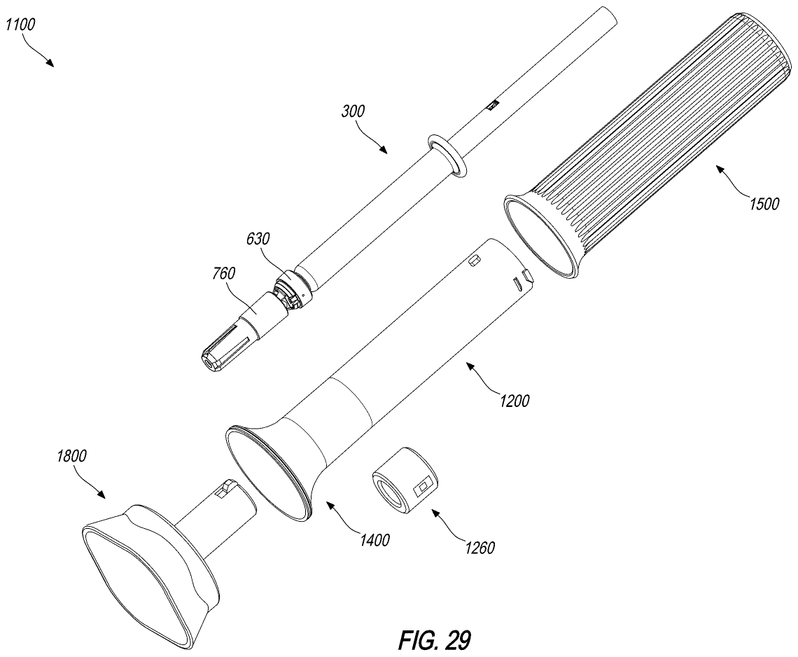

FIGS. 28 and 29 are longitudinal cross-sectional and exploded views of the injection system (1100). The housing member (1200) includes a housing body (1210) defining a housing interior (1220). The injection member (300) is at least partially disposed in the housing interior (1220). Disposing the injection member (300) in the housing member (1200) gives the injection system (1100) an injection pen form factor.

The injection member (300) includes a conventional off-the-shelf pre-filled syringe body (34) defining a medicine reservoir or syringe interior (40). The medicine reservoir or syringe interior (40) is in fluid communication with a syringe needle (620), as shown in FIG. 28. While the injection member (300) is a single chamber injection system, the injection system (1100) can be utilized with multiple chamber injection systems, such as those described in U.S. patent application Ser. No. 18/300,125, which was previously incorporated by reference herein.

As shown in FIG. 28, the injection member (300) includes a needle hub assembly (600) disposed at the distal end of the syringe body (34). The needle hub assembly (600) includes a needle hub (610) and the needle (620), where the needle (620) is coupled to the needle hub (610). The needle hub assembly (600) includes a compression ring (630). The needle (620) is covered by a needle shield (760) for storage and before use of the injection system (1100).

As shown in FIG. 28, the injection member (300) also includes a plunger member (44) inserted into the proximal end of the syringe body (34). The injection member (300) further includes a stopper member (32) coupled to the plunger member (44). The plunger member (44) is configured to be manipulated to insert the stopper member (32) distally in the syringe interior (40) relative to the syringe body (34) to deliver liquid medicine disposed within the syringe interior (40) into the patient via the needle (620). The stopper member (32) may comprise a standard butyl rubber and may be coated, such as with a biocompatible lubricious coating (e.g., PTFE), to facilitate sealing and relative motion characteristics against the associated syringe body structure and material. Medicine delivery to a patient by the injection member (300) is controlled by distal insertion of the plunger member (44) relative to the syringe body (34) to various degrees by a user after the needle (620) is inserted into a patient.

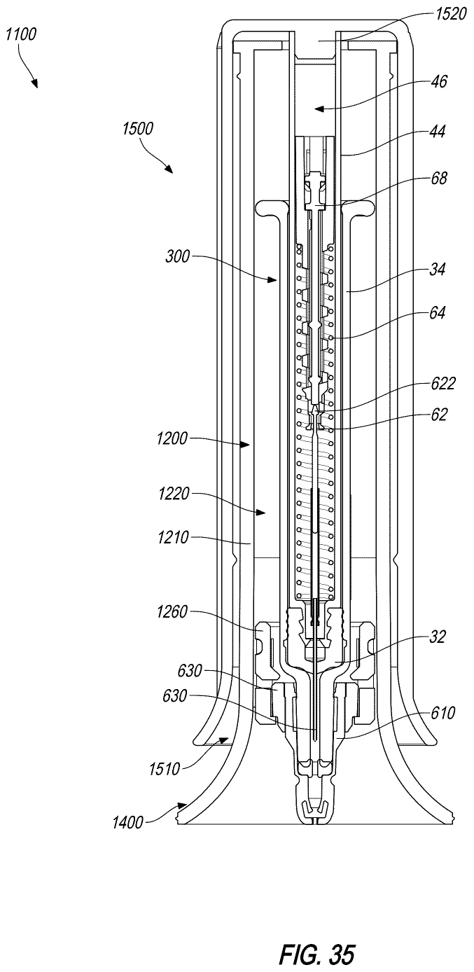

The plunger member (44) defines a plunger interior (46) in which a needle retraction system (60) is substantially disposed. The needle retraction system (60) includes a needle coupling member (62), an energy storage member/spring (64), a spring latch (66), and a latch actuation member (68). The needle coupling member (62) is configured to capture a proximal end member (622) of the needle (620) to operatively couple the needle (620) to the energy storage member/spring (64). The spring latch (66) is configured to temporarily hold the energy storage member/spring (64) in a compressed/energized state as shown in FIG. 28. The latch actuation member (68) is configured to be moved by the proximal end member (622) of the needle (620) to release the energy storage member/spring (64) from the compressed/energized state to transform to an expanded/deenergized state to which the energy storage member/spring (64) is biased (see e.g., FIG. 35). Further details regarding the needle retraction system (60) are described in U.S. patent application Ser. No. 14/696,342, which was previously incorporated by reference herein.

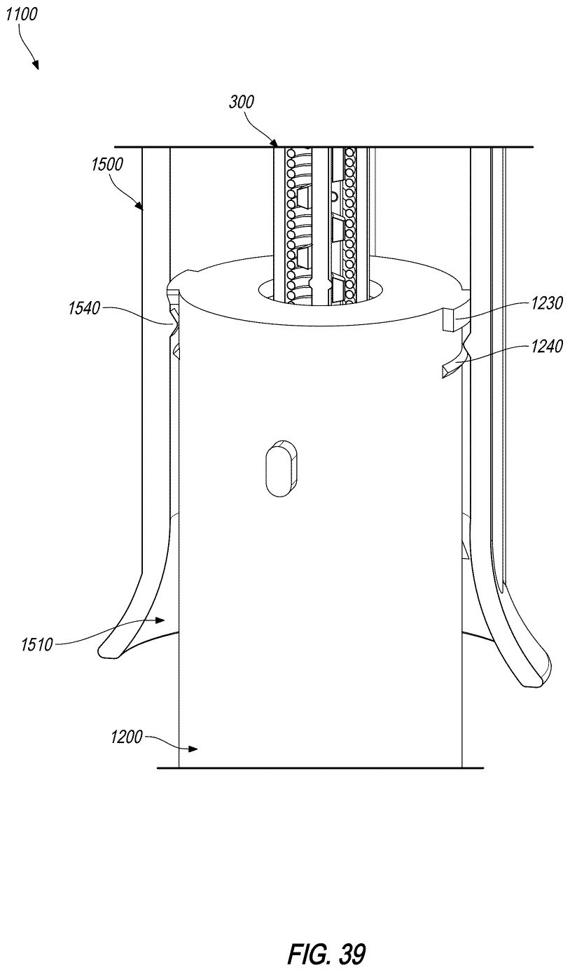

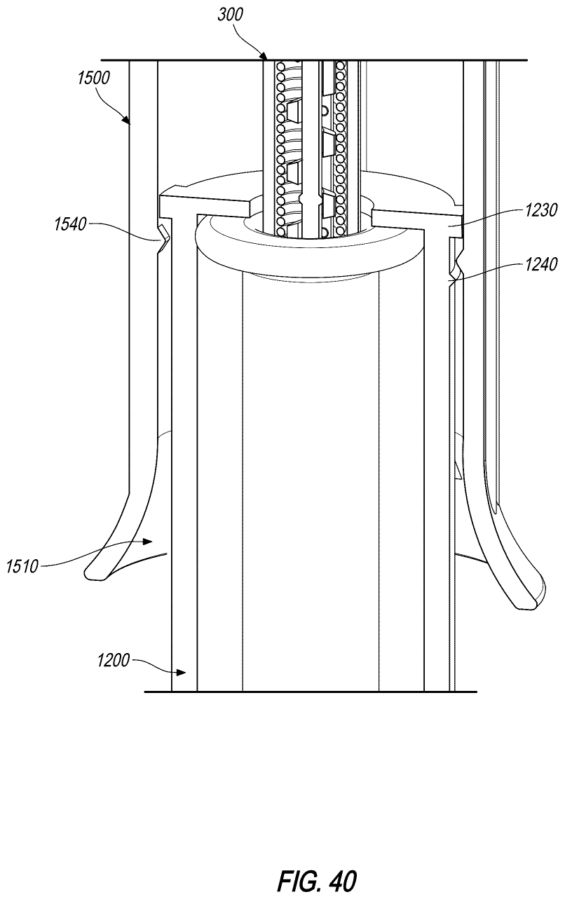

As shown in FIGS. 28 and 29, the injection system (1100) also includes a plunger manipulation member (1500) disposed partially around a proximal end of the housing member (1200). The plunger manipulation member (1500) has an open distal end (1510) disposed around a proximal end of the housing member (1200). The plunger manipulation member (1500) defines a plunger interface (1520) configured to couple to a proximal end of the plunger member (44), which does not have a thumbpad. The plunger manipulation member (1500) also defines a knurled external surface (1530) to facilitate handling of the plunger manipulation member (1500) by the user. The plunger manipulation member (1500) further defines a radially inward extending detent (1540) as shown in FIGS. 39 and 40. The plunger manipulation member (1500) also defines a longitudinal slot (1550) configured to facilitate assembly of the injection system (1100) and to maintain alignment of the radially inward extending detent (1540) of the plunger manipulation member (1500) and the radially outward extending detent (1240) of the housing member (1200) during transport/storage and ready-to-use configurations.

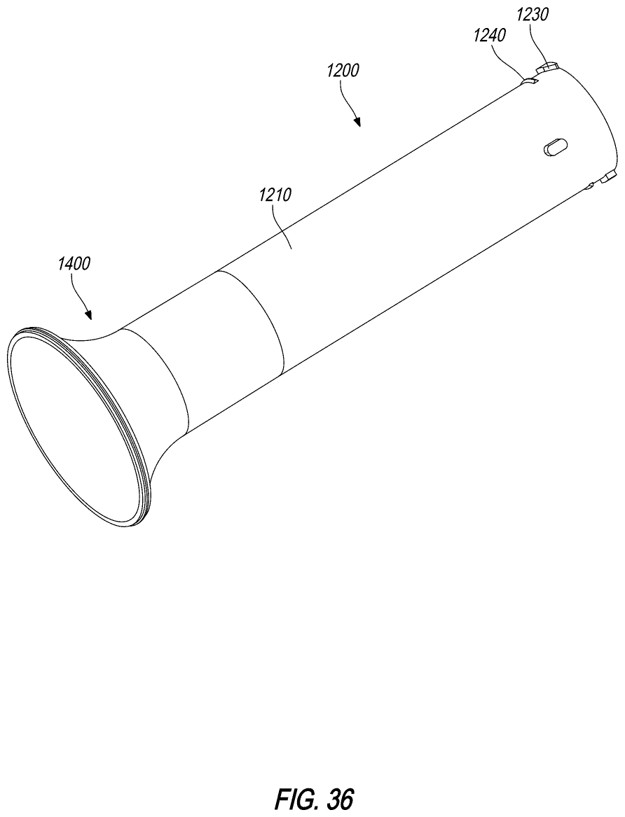

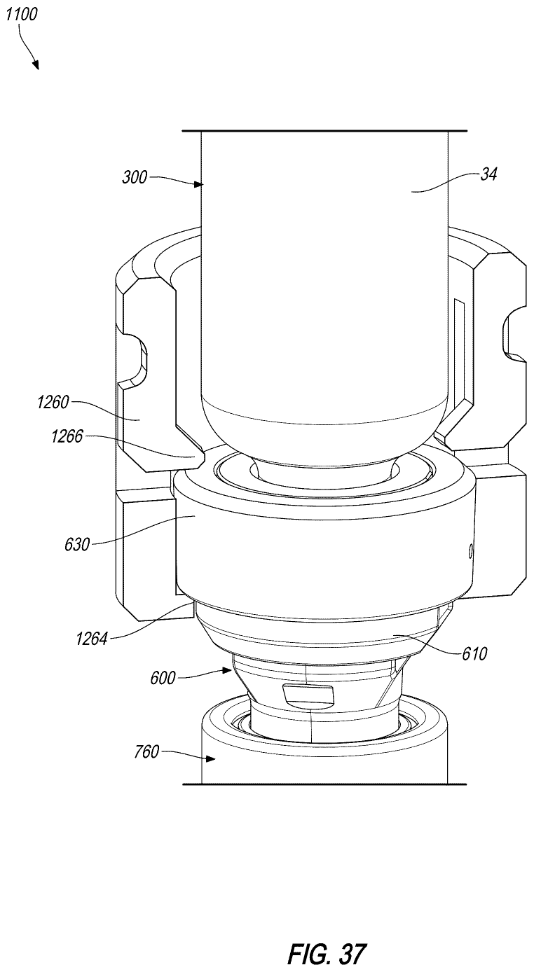

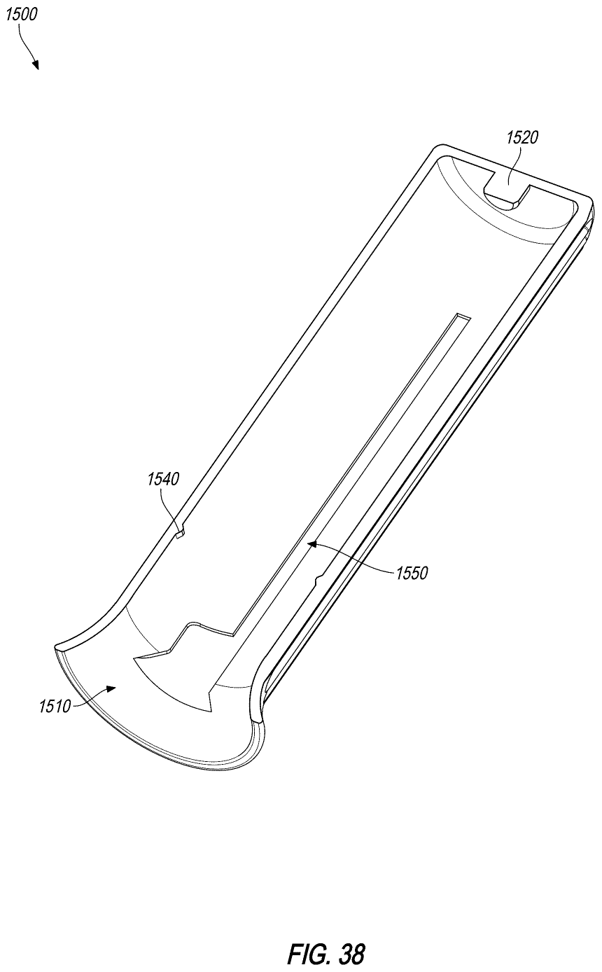

The injection system (1100) further includes a ring (1260) slidably disposed in the housing interior (1220) and configured to center the injection member (300) in the housing member (1200). The ring (1260) defines a distal flange (1264) and a radially inward extending tab (1266) configured to hold the compression ring (630) of the needle hub assembly (600) to fix the injection member (300) to the ring (1260), as shown in FIGS. 28 and 37. The housing member (1200) defines a proximal end radially outward extending flange (1230) and a radially outward extending detent (1240) distal of the proximal end radially outward extending flange (1230) to interfere with the radially inward extending detent (1540) to limit movement of the plunger manipulation member (1500) relative to the housing member (1200), as shown in FIGS. 36, 39, and 40.

The housing member (1200) also includes a needle obscuring member (1400) extending from distal end thereof as shown in FIGS. 28, 29, and 36. The needle obscuring member (1400) includes an outwardly flaring distal end configured to obscure a distal end of the needle (620) from a patient during use. U.S. patent application Ser. No. 18/300,125 and U.S. Provisional Patent Application Ser. No. 63/596,404, which were previously incorporated by reference herein, describe injection system components that have functions (e.g., needle obscuring) similar to those of the needle obscuring member (1400) described herein.

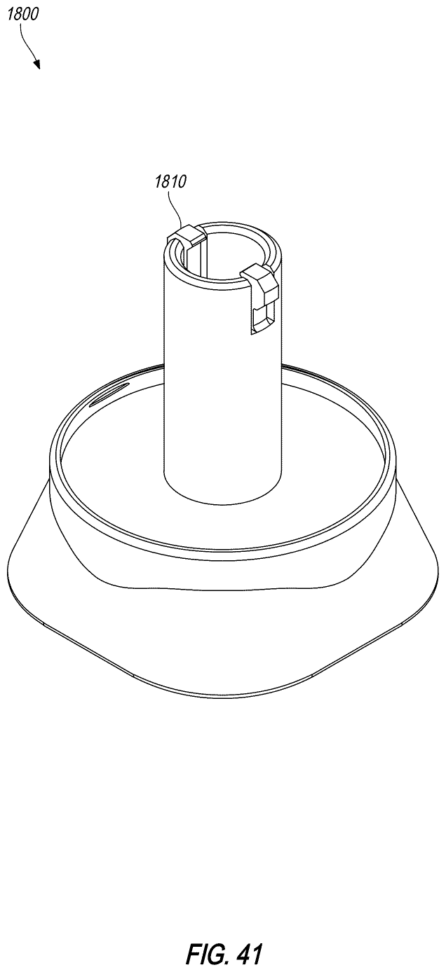

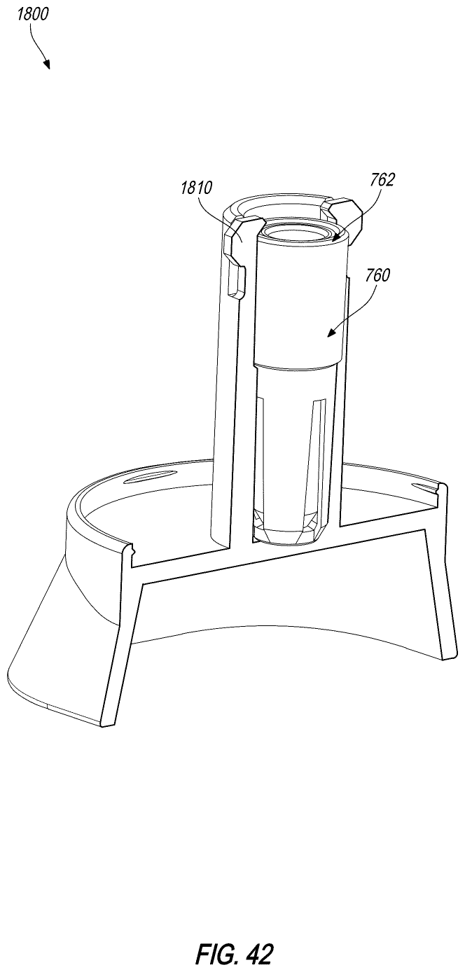

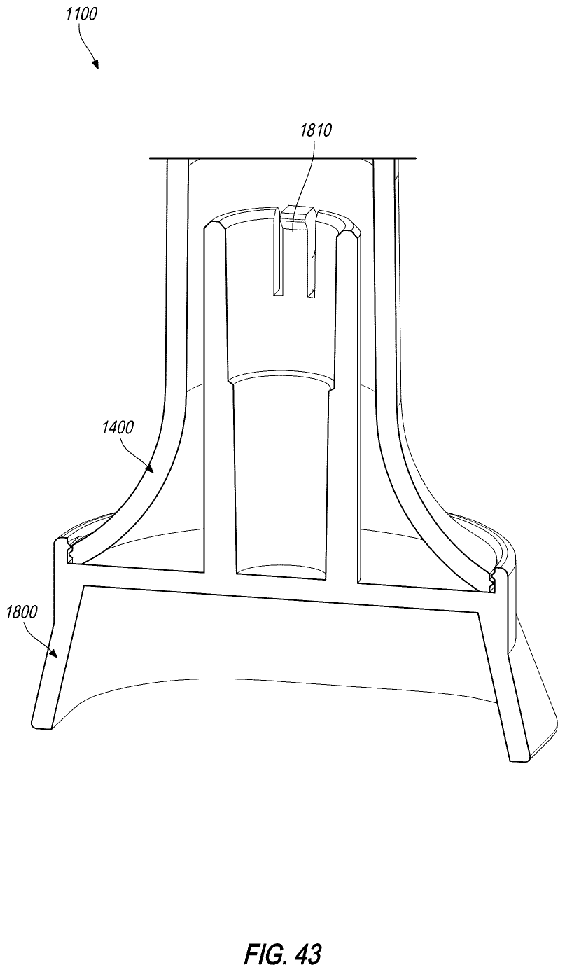

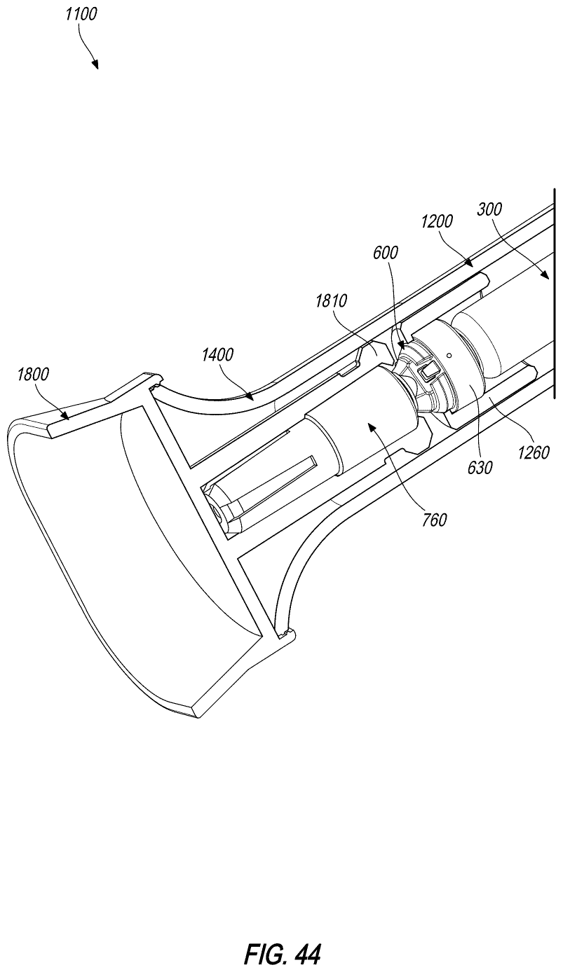

The injection system (1100) further includes a needle shield handling member (1800), which is also shown in FIGS. 41 to 44. The needle shield handling member (1800) is configured to allow a user to remove a needle shield (760) from the needle hub assembly (600) of an injection member (300) disposed inside of a housing member (1200) and a needle obscuring member (1400) such as in the transport/storage configuration depicted in FIGS. 26 to 28, 39, 40, 44, and 45A. The needle shield handling member (1800) defines a pair of flexible tabs (1810) configured to interfere with a proximally facing surface (762) of the needle shield (760) to couple the needle shield handling member (1800) to the needle shield (760). Coupling the needle shield handling member (1800) to the needle shield (760) allows a user to pull the needle shield handling member (1800) distally to remove the needle shield (760) from the needle hub assembly (600) as shown in FIG. 30.

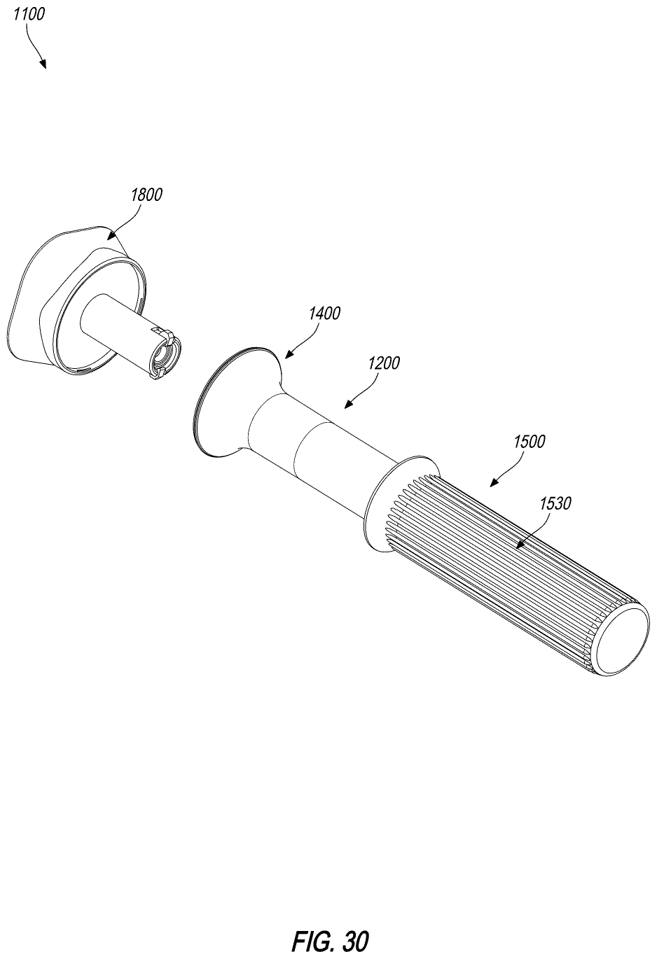

After removing the needle shield handling member (1800) and the needle shield (760) attached thereto, the injection system (1100) depicted in FIGS. 30, 31, and 45B are in a ready-to-use configuration. In the ready-to-use configuration, the needle obscuring member (1400) visually hides the sharp distal end of the needle (620) from the patient. The needle obscuring member (1400) may also tactilely hide the sharp distal end of the needle (620) from the patients by touching the skin of the patient at substantially the same time that the sharp distal end of the needle (620) touches the skin of the patient. In a first step in an injection method, the injection system (1100) in the ready-to-use configuration is disposed in contact with a patient such that the needle obscuring member (1400) and the sharp distal end of the needle (620) is in contact with the skin of the patient.

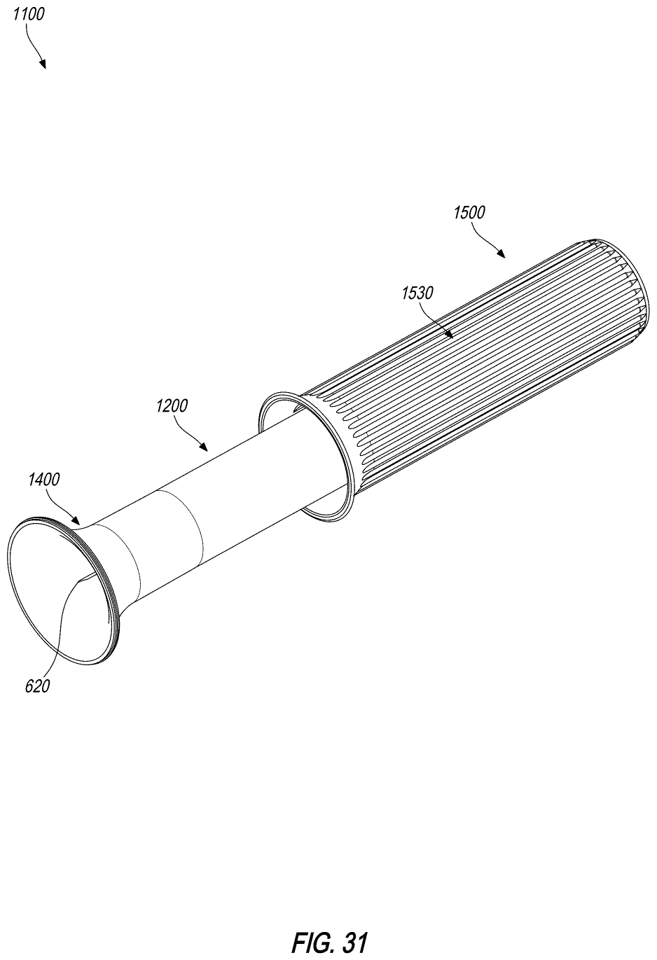

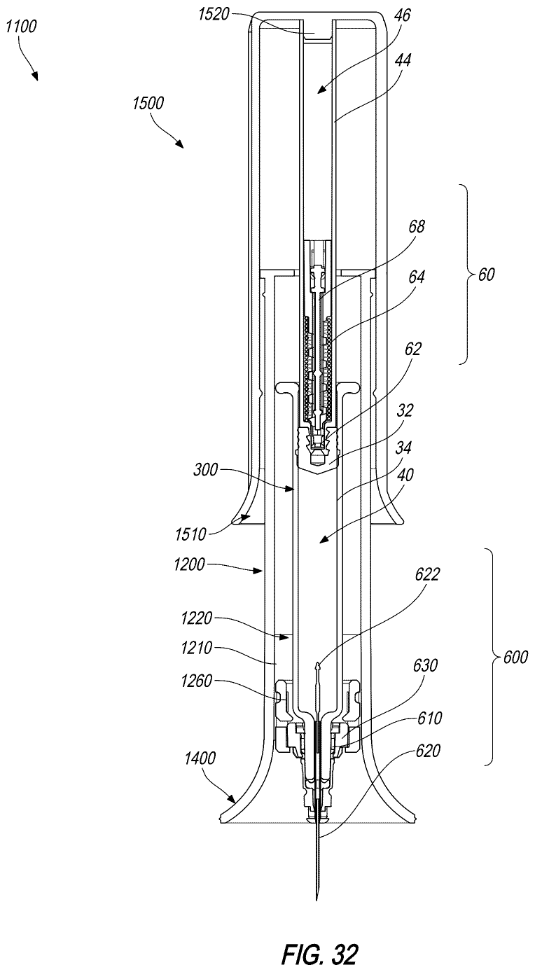

In the next step in the injection method, with needle obscuring member (1400) and the sharp distal end of the needle (620) of the injection system (1100) in contact with the skin of the patient, distally directed force is applied to the plunger manipulation member (1500), which is grasped in a hand of a user to first slide the injection member (300) distally in the housing interior (1220) to insert the needle (620) into the patient. Inserting the needle (620) into the patient transforms the injection system (1100) from the ready-to-use configuration depicted in FIGS. 30, 31, and 45B to the extended configuration depicted in FIGS. 32, 33, and 45C.

In the transport/storage configuration depicted in FIGS. 26 to 28, 39, 40, 44, and 45A and the ready-to-use configuration depicted in FIGS. 30, 31, and 45B, the proximal end radially outward extending flange (1230) and a radially outward extending detent (1240) are disposed on either side of the radially inward extending detent (1540) to limit movement of the plunger manipulation member (1500) relative to the housing member (1200), as shown in FIGS. 39 and 40. Sufficient distally directed force applied to the plunger manipulation member (1500) can overcome the interference between the radially outward extending detent (1240) and the radially inward extending detent (1540) to allow distal movement of the plunger manipulation member (1500) relative to the housing member (1200) to transform the injection system (1100) from the ready-to-use configuration to the extended configuration.

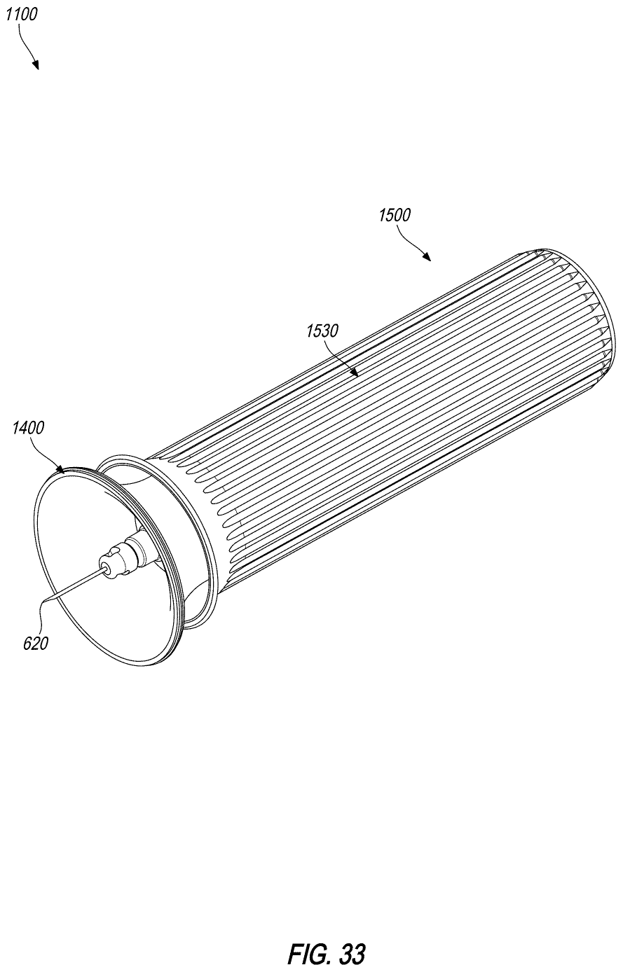

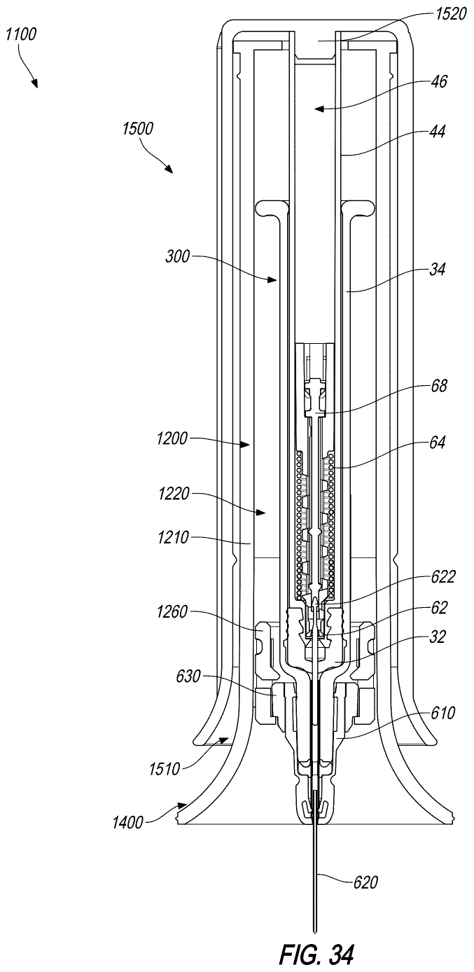

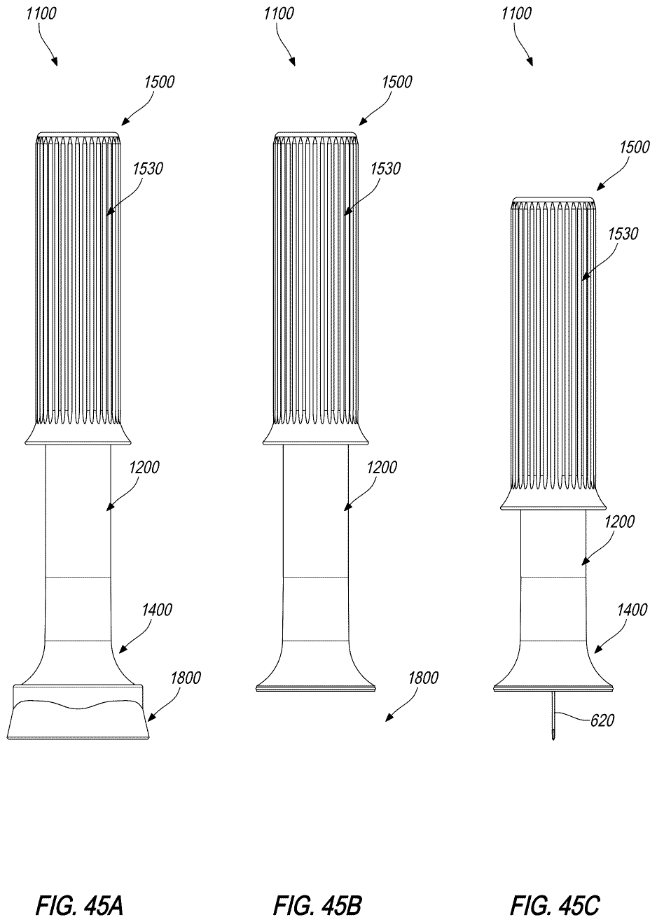

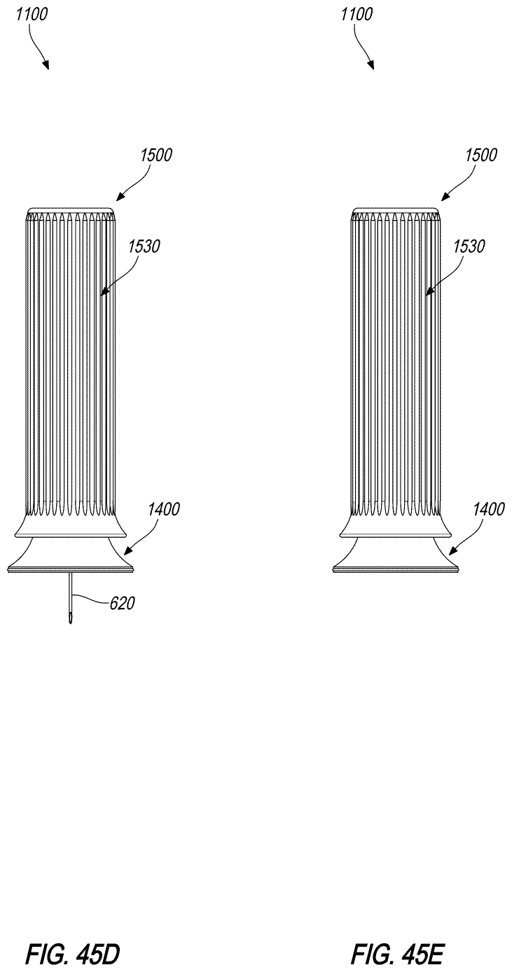

In the next step in the injection method, with the needle (620) inserted into a patient, distally directed force is continued to be applied to the plunger manipulation member (1500) to move the plunger manipulation member (1500), the plunger member (44) attached thereto, and the stopper member (32) attached to the plunger member (44) distally. Moving the stopper member (32) distally in the syringe interior (40) ejects a liquid medicine from the syringe interior (40) through the needle (620) and into the patients. Applying sufficient distally directed force to the plunger manipulation member (1500) to move the syringe member (32) to a distal end of the syringe body (34) completes the injection and converts the injection system (1100) from the extended configuration depicted in FIGS. 32, 33, and 45C to the injection-complete configuration depicted in FIGS. 34 and 45D.

From the injection-complete configuration depicted in FIGS. 34 and 45D, continued application of distally directed force to the plunger manipulation member (1500) both causes the proximal end member (622) of the needle (620) to be captured by/coupled to the needle coupling member (62) and moves the latch actuation member (68) to release the spring latch (66) thereby allowing the energy storage member/spring (64) to transform from the compressed/energized state to transform to an expanded/deenergized state. When transforming from the compressed state to the expanded state, the energy storage member/spring (64) pulls the needle coupling member (62) and the needle (620) attached thereto proximally into the injection system (1100) until the sharp distal end of the needle (620) is disposed in the needle hub (610) and a distal end of the syringe body (34), thereby rendering the injection system (1100) safe for disposal. Retracting the needle after injection transforms the injection system (1100) from the injection-complete configuration depicted in FIGS. 34 and 45D to the needle-retracted/safe configuration depicted in FIGS. 35 and 45E.

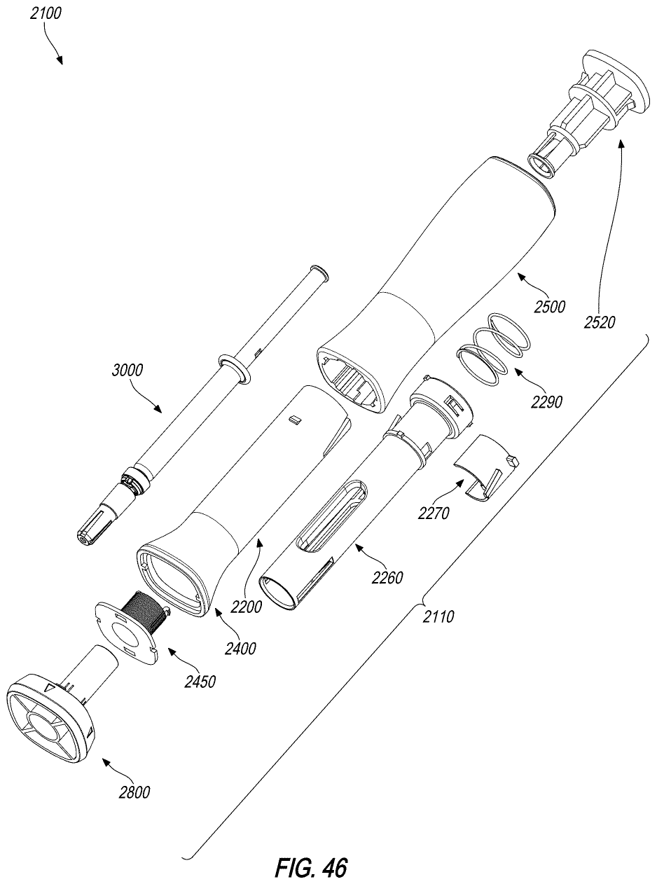

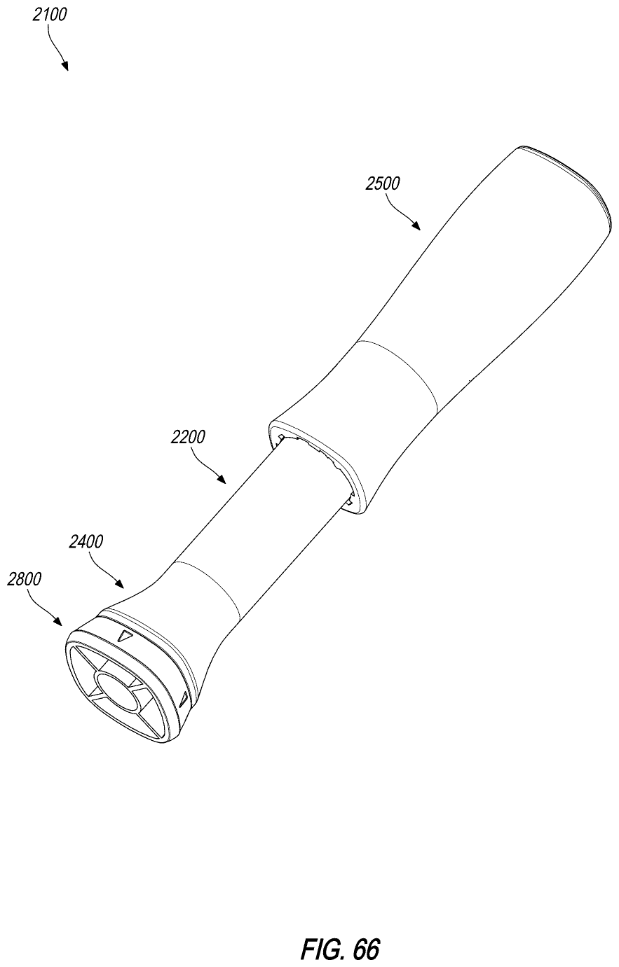

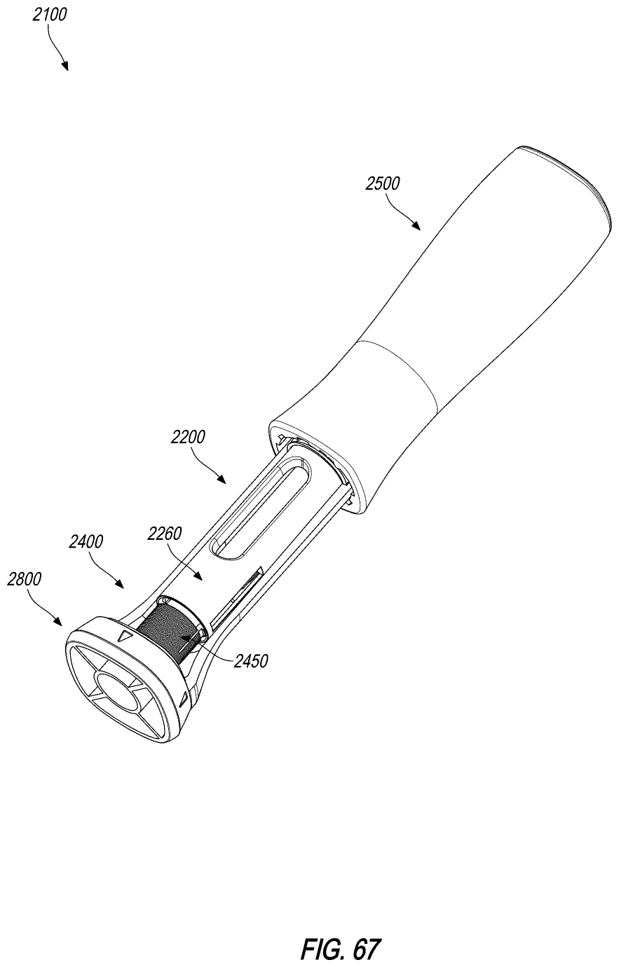

FIG. 46 is a partially exploded view of an injection system (2100), according to some embodiments. FIGS. 66 to 68 are perspective, partial cutaway perspective, and longitudinal cross-sectional views of the injection system (2100) in a transport/storage configuration according to some embodiments. The injection system (2100) includes a housing member (2110) and an injection member (300). The housing member (2100) includes a handle (2500), an outer tube (2200), a syringe housing (2260), a shroud (2450), and a needle shield handling member (2800). The handle (2500) is disposed partially around a proximal end of the outer tube (2200). The syringe housing (2260) is disposed partially in the outer tube (2200). The injection system (2100) also includes an injection member (300) at least partially disposed in the syringe housing (2260). The outer tube (2200) includes a needle obscuring member (2400) at a distal end thereof. The needle obscuring member (2400) is a flared portion at a distal end of the housing member (2200). The housing member (2110) also includes a handle cap (2520) disposed partially in a proximal end of the handle (2500). The housing member (2110) further includes a rotating cuff (2270) disposed around a proximal end of the syringe housing (2260). Moreover, the housing member (2110) includes a needle insertion spring (2290) around the syringe housing (2260) distal of the rotating cuff (2270).

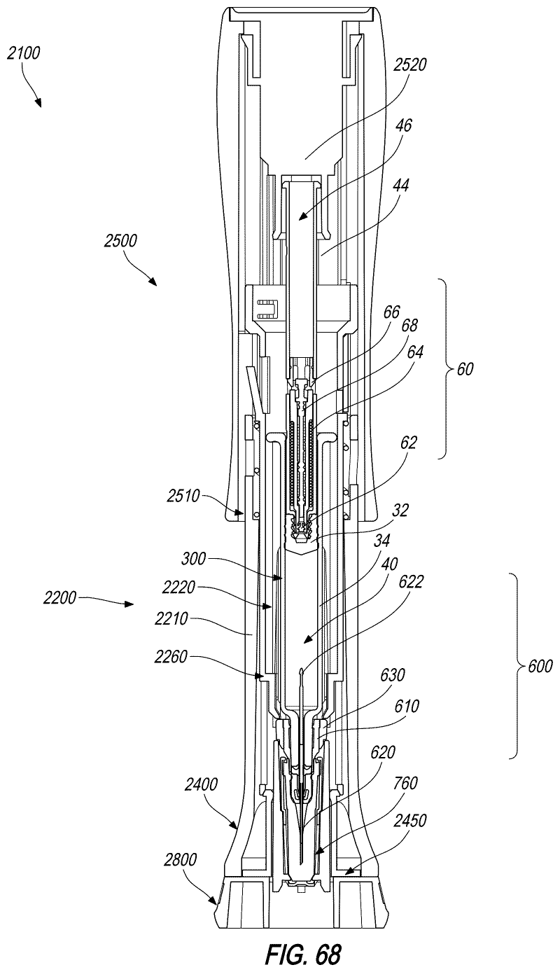

As shown in FIG. 68, the outer tube (2200) includes a tubular body (2210) defining an outer tube interior (2220). The injection member (300) is at least partially disposed in the outer tube interior (2220). Disposing the injection member (300) in the outer tube (2200) gives the injection system (2100) an injection pen form factor.

The injection member (300) includes a conventional off-the-shelf pre-filled syringe body (34) defining a medicine reservoir or syringe interior (40). The medicine reservoir or syringe interior (40) is in fluid communication with a syringe needle (620), as shown in FIG. 68. While the injection member (300) is a single chamber injection system, the injection system (2100) can be utilized with multiple chamber injection systems, such as those described in U.S. patent application Ser. No. 18/300,125, which was previously incorporated by reference herein.

As shown in FIG. 68, the injection member (300) includes a needle hub assembly (600) disposed at the distal end of the syringe body (34). The needle hub assembly (600) includes a needle hub (610) and the needle (620), where the needle (620) is coupled to the needle hub (610). The needle hub assembly (600) includes a compression ring (630). The needle (620) is covered by a needle shield (760) for storage and before use of the injection system (2100).

As shown in FIG. 68, the injection member (300) also includes a plunger member (44) inserted into the proximal end of the syringe body (34). The injection member (300) further includes a stopper member (32) coupled to the plunger member (44). The plunger member (44) is configured to be manipulated to insert the stopper member (32) distally in the syringe interior (40) relative to the syringe body (34) to deliver liquid medicine disposed within the syringe interior (40) into the patient via the needle (620). The stopper member (32) may comprise a standard butyl rubber and may be coated, such as with a biocompatible lubricious coating (e.g., PTFE), to facilitate sealing and relative motion characteristics against the associated syringe body structure and material. Medicine delivery to a patient by the injection member (300) is controlled by distal insertion of the plunger member (44) relative to the syringe body (34) to various degrees by a user after the needle (620) is inserted into a patient.

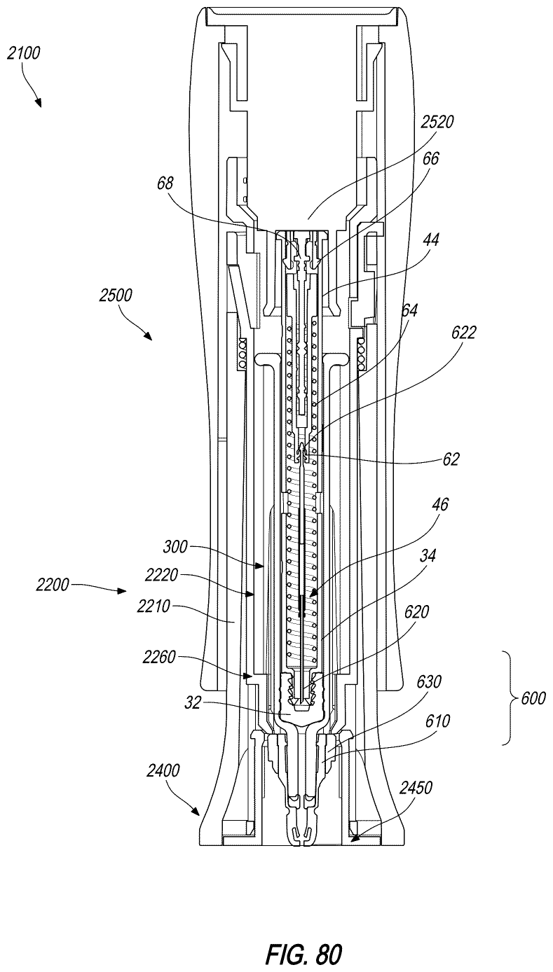

The plunger member (44) defines a plunger interior (46) in which a needle retraction system (60) is substantially disposed. The needle retraction system (60) includes a needle coupling member (62), an energy storage member/spring (64), a spring latch (66), and a latch actuation member (68). The needle coupling member (62) is configured to capture a proximal end member (622) of the needle (620) to operatively couple the needle (620) to the energy storage member/spring (64). The spring latch (66) is configured to temporarily hold the energy storage member/spring (64) in a compressed/energized state as shown in FIG. 68. The latch actuation member (68) is configured to be moved by the proximal end member (622) of the needle (620) to release the energy storage member/spring (64) from the compressed/energized state to transform to an expanded/deenergized state to which the energy storage member/spring (64) is biased (see e.g., FIG. 80). Further details regarding the needle retraction system (60) are described in U.S. patent application Ser. No. 14/696,342, which was previously incorporated by reference herein.

As shown in FIGS. 66 to 68, the injection system (2100) also includes a handle (2500) disposed partially around a proximal end of the outer tube (2200). The handle (2500) has an open distal end (2510) disposed around a proximal end of the outer tube (2200). The handle (2500) defines a handle cap (2520) configured to couple to a proximal end of the plunger member (44), which does not have a thumbpad.

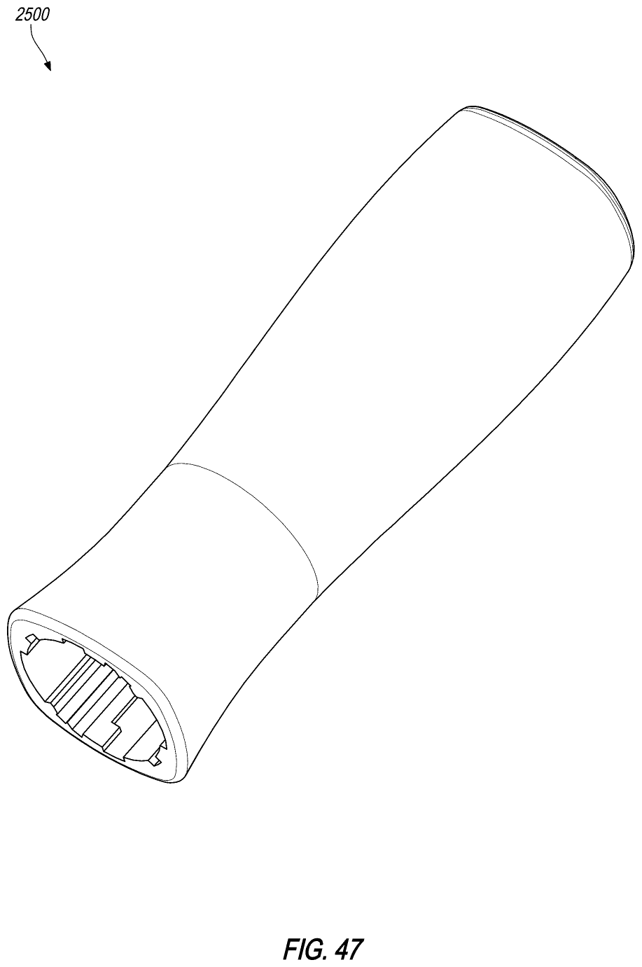

The outer tube (2200) also includes a needle obscuring member (2400) extending from distal end thereof as shown in FIGS. 47 and 66 to 68. The needle obscuring member (2400) includes an outwardly flaring distal end configured to obscure a distal end of the needle (620) from a patient during use. U.S. patent application Ser. No. 18/300,125 and U.S. Provisional Patent Application Ser. No. 63/596,404, which were previously incorporated by reference herein, describe injection system components that have functions (e.g., needle obscuring) similar to those of the needle obscuring member (2400) described herein.

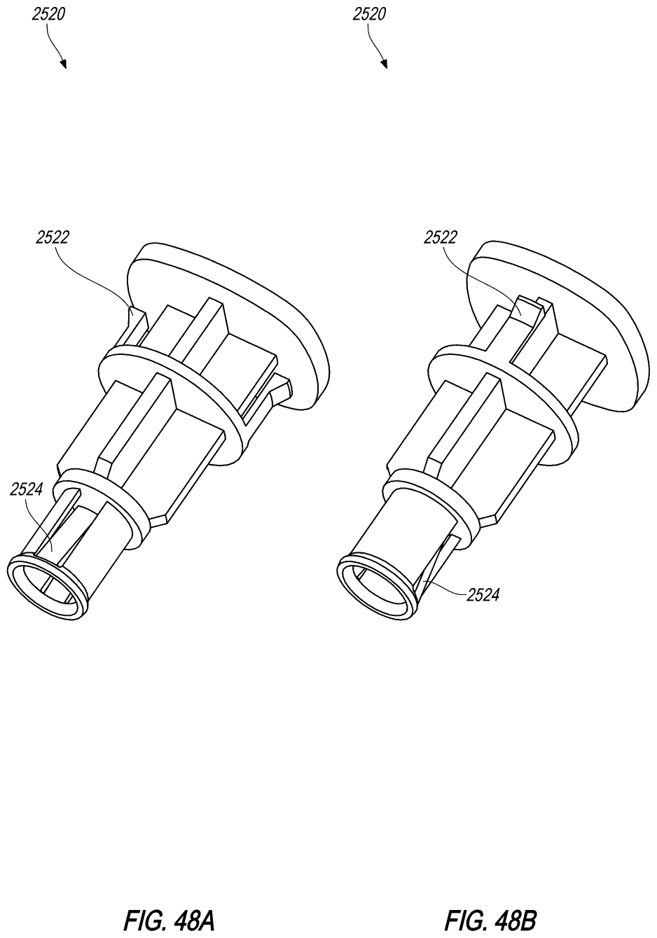

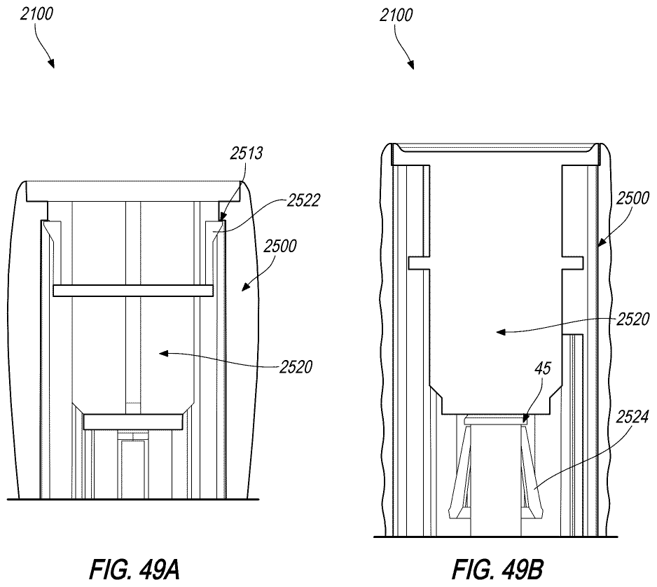

FIG. 47 is a perspective view of the handle (2500) configured for use with the injection system (2100), according to some embodiments. FIGS. 48A and 48B are perspective views of the handle cap (2520) configured for use with the injection system (2100), according to some embodiments. FIGS. 49A and 49B are detailed perspective views of the injection system (2100), including the handle (2500) and the handle cap (2520), according to some embodiments. As shown in FIGS. 48A and 48B, the handle cap (2520) defines a pair of handle cap latches (2522) at a proximal end thereof and a pair of plunger member arms (2524) at a distal end thereof. As shown in FIGS. 49A and 49B, the handle cap (2520) is disposed in and closes an open proximal end of the handle (2500).

As shown in FIG. 49A, the handle (2500) defines a proximal end wall (2513) on an inner surface thereof. The proximal end wall (2513) is configured to interfere with the handle cap latches (2522) to prevent the handle cap (2520) from moving proximally relative to the handle (2500) to couple the handle cap (2520) to the handle (2500). As shown in FIG. 49B, the plunger member (44) defines a distally facing surface (45) at a proximal end thereof. The distally facing surface (45) is configured to interfere with the plunger member arms (2524) to prevent the handle (2500) from moving proximally relative to the plunger member (44) to couple the plunger member (44) to the handle cap (2520) and handle (2500).

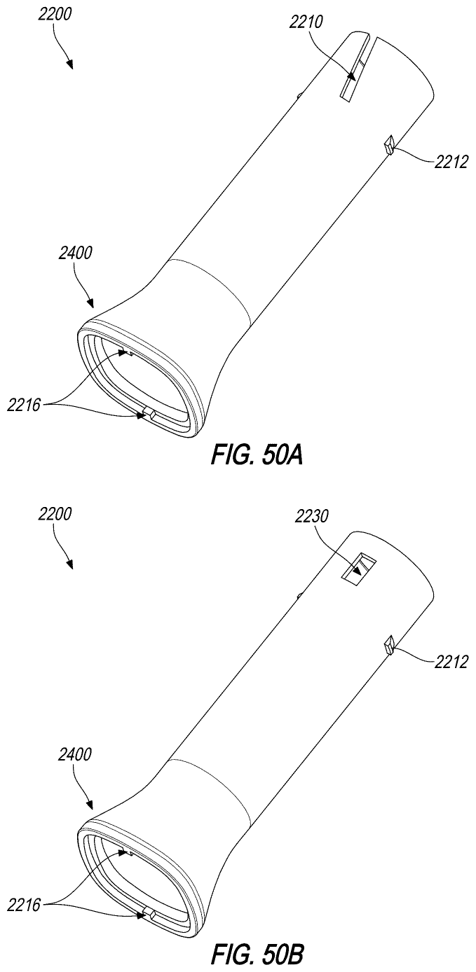

FIGS. 50A and 50B are perspective views of the outer tube (2200) configured for use with the injection system (2100), according to some embodiments. The outer tube (2200) defines an angled slot (2210), a pair of outer tube radially outward extending tabs (2212), and an anti-return window (2230) at a proximal end thereof. The angled slot (2210) is disposed on an opposite side of the outer tube (2200) from the anti-return window (2230). The pair of outer tube radially outward extending tabs (2212) are disposed opposite of each other and 90 degrees offset from the angled slot (2210) and the anti-return window (2230). The outer tube (2200) also defines a needle obscuring member (2400) and a pair of tabs (2216) at a distal end thereof.

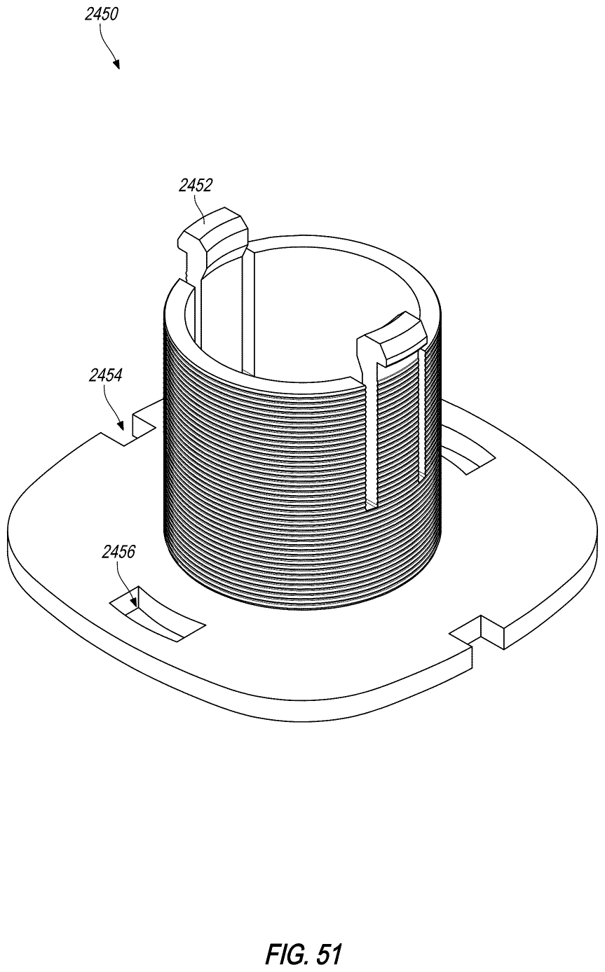

FIG. 51 is a perspective view of the shroud (2450) configured for use with the injection system (2100), according to some embodiments. The shroud (2450) defines a pair of shroud arms (2452) at a proximal end thereof. The shroud also defines a pair of shroud slots (2454) and a pair of shroud openings (2456) at a distal end thereof. The respective pairs of shroud arms (2452), shroud slots (2454), and shroud openings (2456) are disposed opposite of each other. The pair of shroud openings (2456) are disposed 90 degrees offset from the pairs of shroud arms (2452) and shroud slots (2454). The pair of shroud slots (2454) cooperate with the pair of tabs (2216) at the distal end of the outer tube (2200) to align the shroud (2450), as the shroud (2450) is fixed to the outer tube (2200).

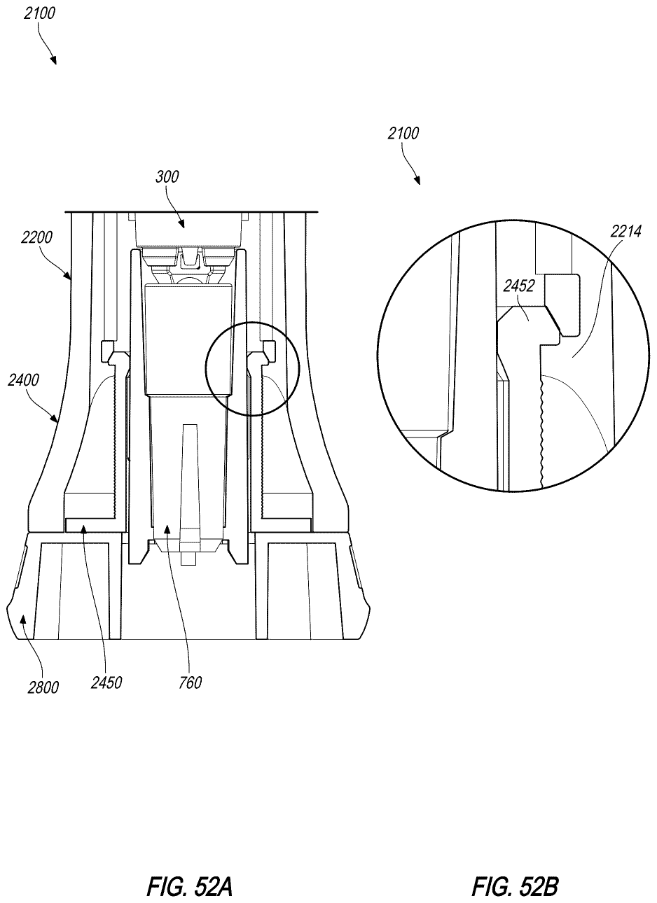

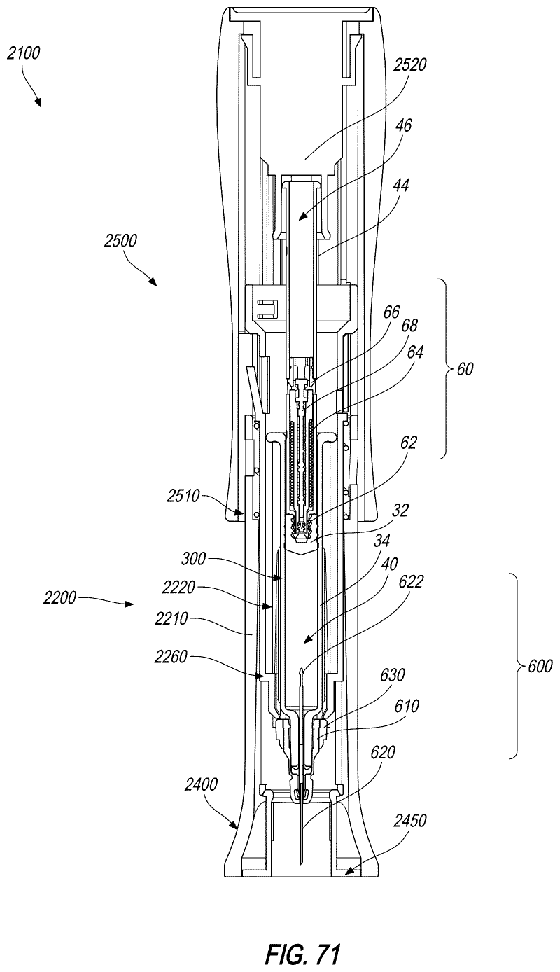

FIGS. 52A and 52B are detailed and more detailed longitudinal cross-sectional views of the distal end of the injection system (2100), according to some embodiments. As shown in FIG. 52A, when the needle shield cap (2800) and the needle shield (760) are removably coupled to the shroud (2450) and the needle hub (610) (see, e.g., FIG. 68) respectively, the needle shield cap (2800) interferes with the shroud arms (2452) to maintain a radial position of the shroud arms (2452) relative to the distal end (2214) of the syringe housing (2260). This interference prevents the shroud arms (2452) from flexing radially inward, thereby preventing the syringe housing (2260) and the injection member (300) contained in the syringe housing (2260) from moving distally relative to the shroud (2450) and the outer tube (2200) until the needle shield cap (2800) and the needle shield (760) are removed from the needle hub (610) (see, e.g., FIG. 71). The shroud arms (2452) prevent the syringe housing (2260) and the injection member (300) from sliding distally during removal of the needle shield cap (2800) and the needle shield (760) to prevent the needle from being prematurely exposed. After the needle shield cap (2800) and the needle shield (760) are fully removed (see, e.g., FIG. 71), the shroud arms (2452) are able to flex radially inward to into an open distal end of the syringe housing (2260) to allow the syringe housing (2260) and the injection member (300) to move distally to insert the needle (620) into tissue when a predetermined amount of force (configured to be provided by a user) is applied to the handle (2500).

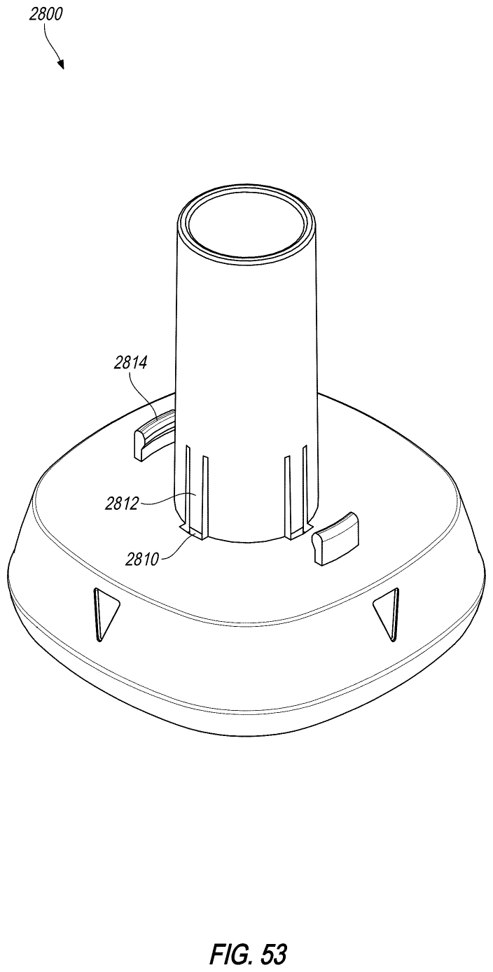

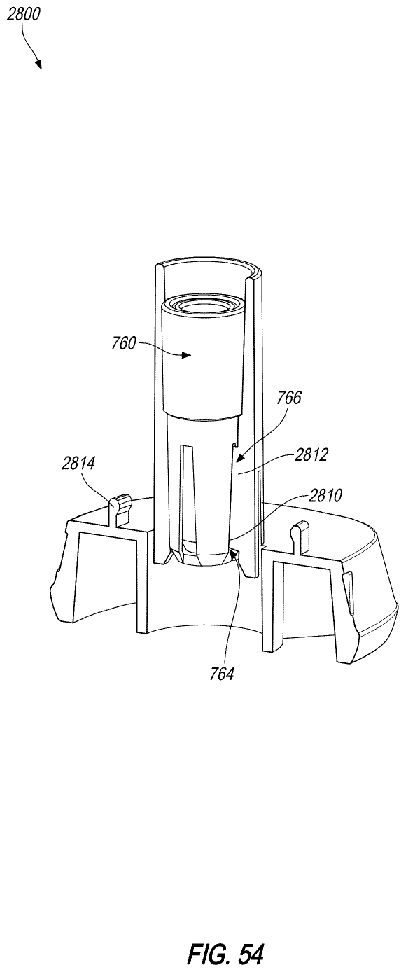

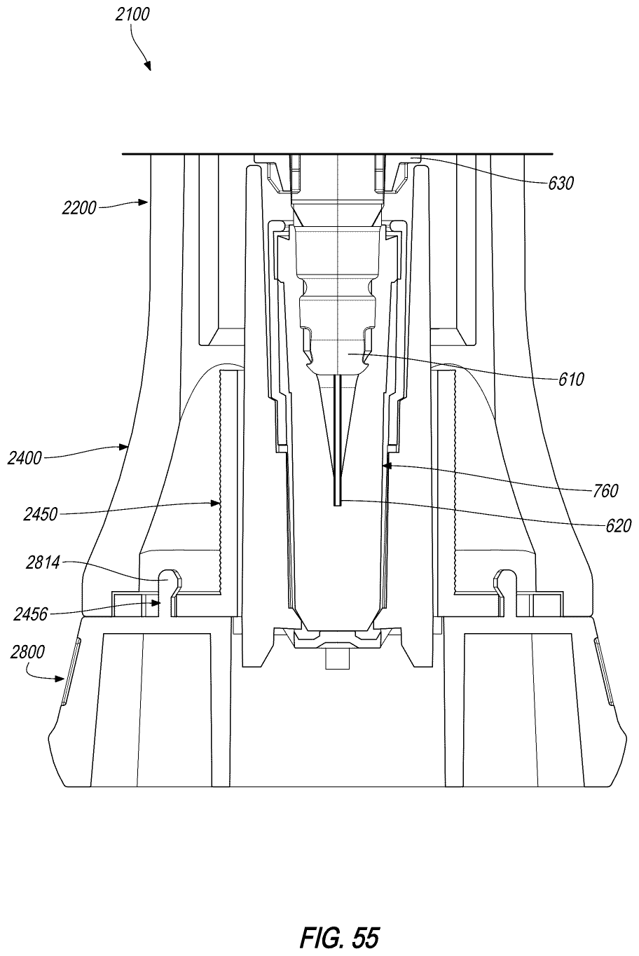

FIGS. 53 and 54 are perspective and partial cutaway perspective views of the needle shield cap (2800) configured for use with the injection system (2100), according to some embodiments. The needle shield (760) defines a plurality of needle shield distal openings (764) at a distal end of the needle shield (760), and a plurality of needle shield longitudinal slots (766) along a radially outward surface of the needle shield (760). The needle shield cap (2800) defines a plurality (e.g., four) of needle shield cap latches (2810) configured to cooperate with a respective plurality of needle shield distal openings (764) to couple the needle shield cap (2800) to the needle shield (760). The needle shield cap (2800) also defines a plurality (e.g., two) of needle shield cap guide columns (2812) configured to cooperate with a respective plurality of needle shield longitudinal slots (766) to couple the needle shield cap (2800) to the needle shield (760). The plurality of needle shield cap guide columns (2812) also aligns the needle shield (760) in the needle shield cap (2800). The needle shield cap (2800) further defines a pair of needle shield cap detents (2814) configured to cooperate with the corresponding pair of shroud openings (2456) to removably couple the needle shield cap (2800) to the shroud (2450) and the outer tube (2200), as shown in FIG. 55.

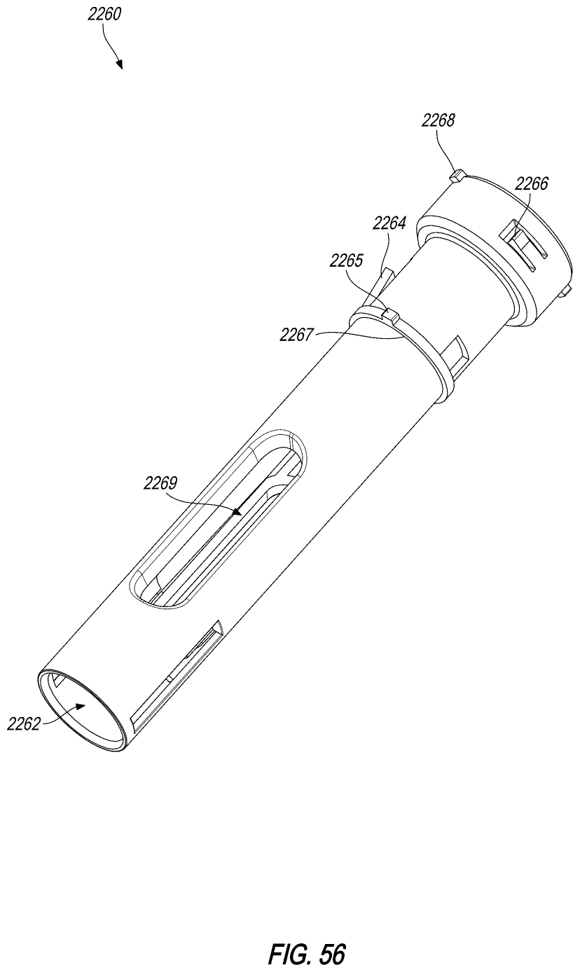

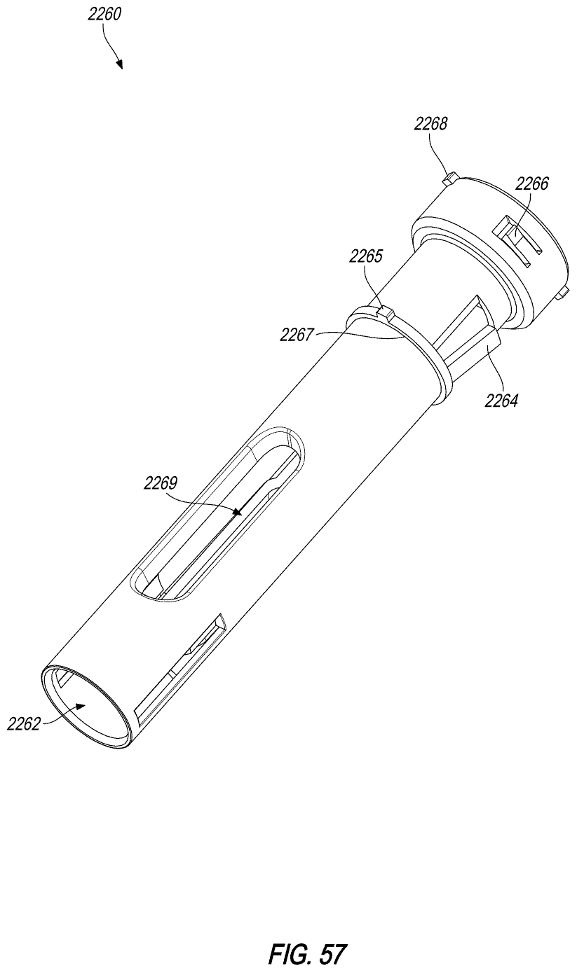

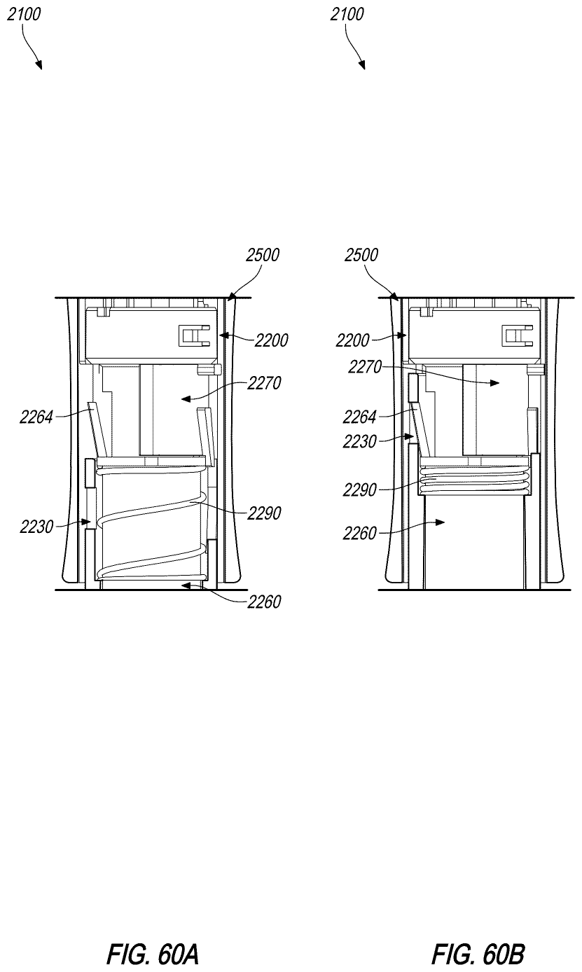

FIGS. 56 and 57 are perspective views of the syringe housing (2260) configured for use with the injection system (2100), according to some embodiments. The syringe housing (2260) defines a pair of syringe housing external housing latches (2266) and a pair of syringe housing radially outward extending tabs (2268) at a proximal end thereof. The respective pairs of syringe housing external housing latches (2266) and syringe housing radially outward extending tabs (2268) are disposed opposite of each other. The syringe housing (2260) also defines a syringe housing anti-return latch (2264) at a proximal end thereof distal of the pair of syringe housing external housing latches (2266). The syringe housing (2260) further defines a pair of syringe housing spring tabs (2265) and a syringe housing flange (2267) configured to interfere with a proximal end of the needle insertion spring (2290) (see FIGS. 60A to 62B). The pair of syringe housing spring tabs (2265) are disposed opposite of each other. Moreover, the syringe housing (2260) defines a syringe housing window (2269) configured to allow the pair of syringe housing spring tabs (2265) to be visible from outside of the syringe housing (2260).

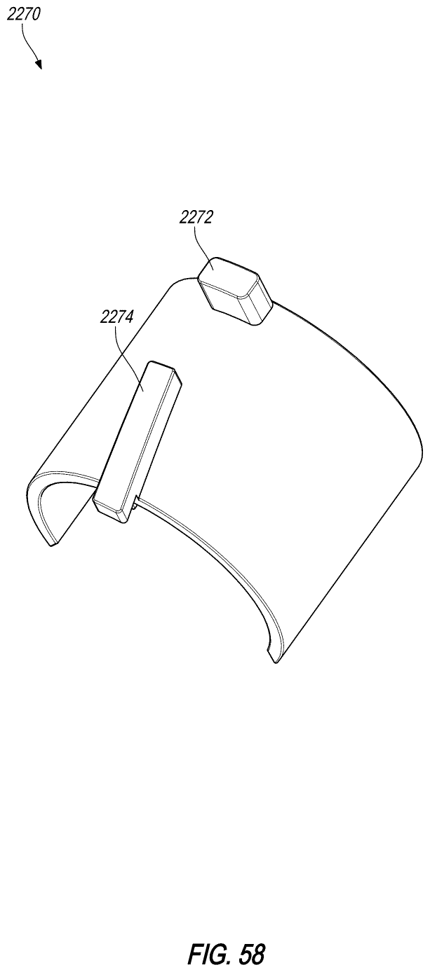

FIG. 58 is a perspective view of the rotating cuff (2270) configured for use with the injection system (2100), according to some embodiments. The rotating cuff (2270) defines a radially extending block (2272) at a proximal end thereof, and an angled block (2274) at a distal end thereof. The radially extending block (2272) and the angled block (2274) are rotationally aligned with each other. The rotating cuff (2270) is disposed around a proximal portion of the syringe housing (2260) and translationally coupled to the syringe housing (2260). However, the rotating cuff (2270) is free to rotate around the syringe housing (2260).

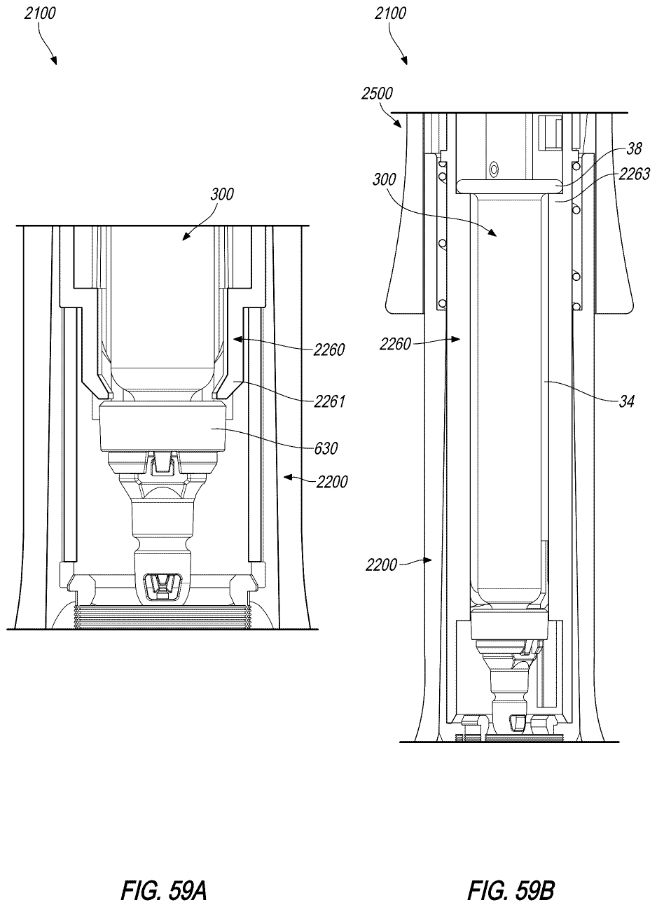

FIGS. 59A and 59B are detailed longitudinal cross-sectional views of the injection system (2100), according to some embodiments. As shown in FIG. 59A, the syringe housing (2260) defines a pair of syringe housing latches (2261) adjacent a distal end thereof. As shown in FIG. 59B, the syringe housing (2260) also defines a circular syringe housing rib (2263) adjacent a proximal end thereof. The syringe housing latch (2261) and the syringe housing rib (2263) are configured to limit longitudinal movement of the injection member (300) relative to the syringe housing (2260). The pair of syringe housing latches (2261) are disposed opposite of each other and flex around the compression ring (630) as the injection member (300) is inserted distally into the syringe housing (2260) and snap over a proximal end of the compression ring to prevent the injection member (300) from moving proximally relative to the syringe housing (2260). The circular syringe housing rib (2263) interferes with a syringe flange (38) at a proximal end of the syringe body (34) to prevent the injection member (300) from moving distally relative to the syringe housing (2260).

FIGS. 60A and 60B are detailed longitudinal cross-sectional views of the injection system (2100), according to some embodiments. FIG. 60A depicts a first state of the injection system (2100) before the distal end of the syringe housing (2260) is pushed distally past the pair of shroud arms (2452). With the injection system (2100) in the first state, the needle insertion spring (2290) is in an expanded state. FIG. 60B depicts a second state of the injection system (2100) after the distal end of the syringe housing (2260) is pushed distally past the shroud arms (2452). With the injection system (2100) in the second state, the needle insertion spring (2290) is in a compressed state. As the needle insertion spring (2290) transitions from the expanded state to the compressed state, the needle insertion spring (2290) acts to provide resistance for a smoother user experience while the needle (620) is being inserted into tissue of a patient. After the distal end of the syringe housing (2260) is pushed past the shroud arms (2452), the needle insertion spring (2290) compresses until the needle (620) is fully inserted into target tissue to provide a similar resistance on the handle (2500) as the injection force, thereby ensuring a smooth force profile over the needle insertion and injection processes.

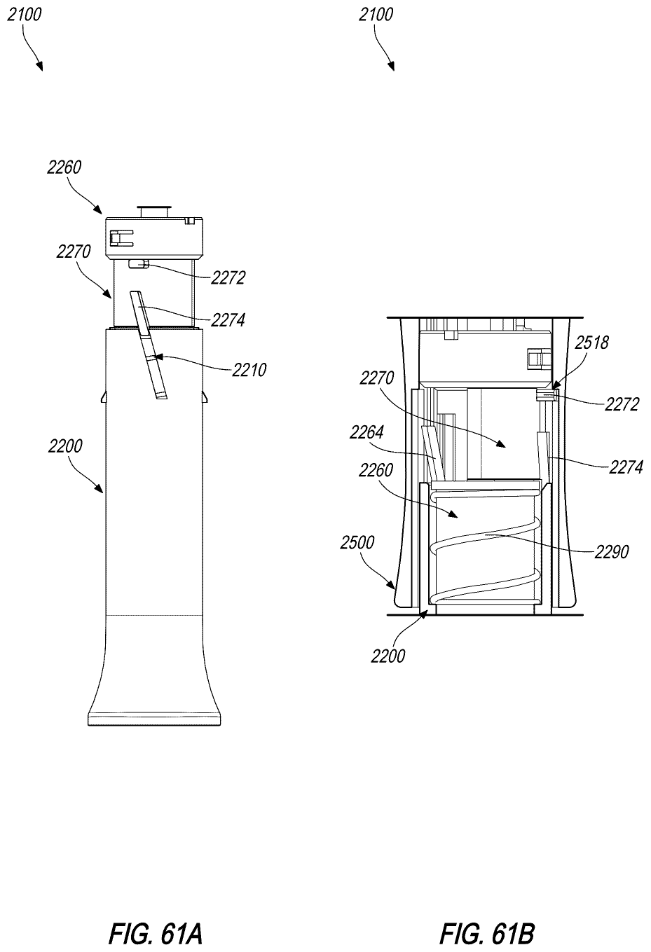

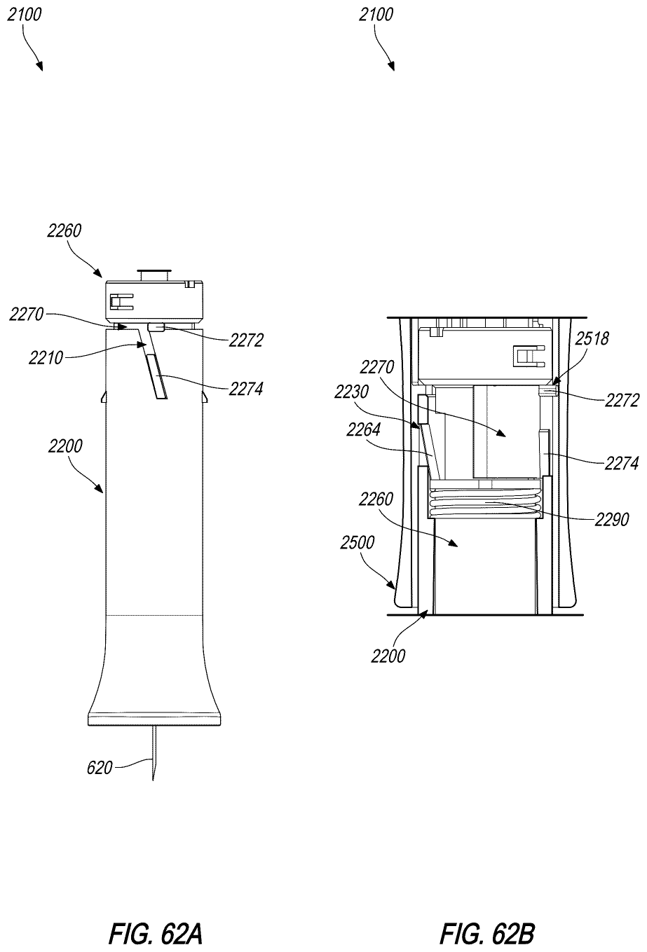

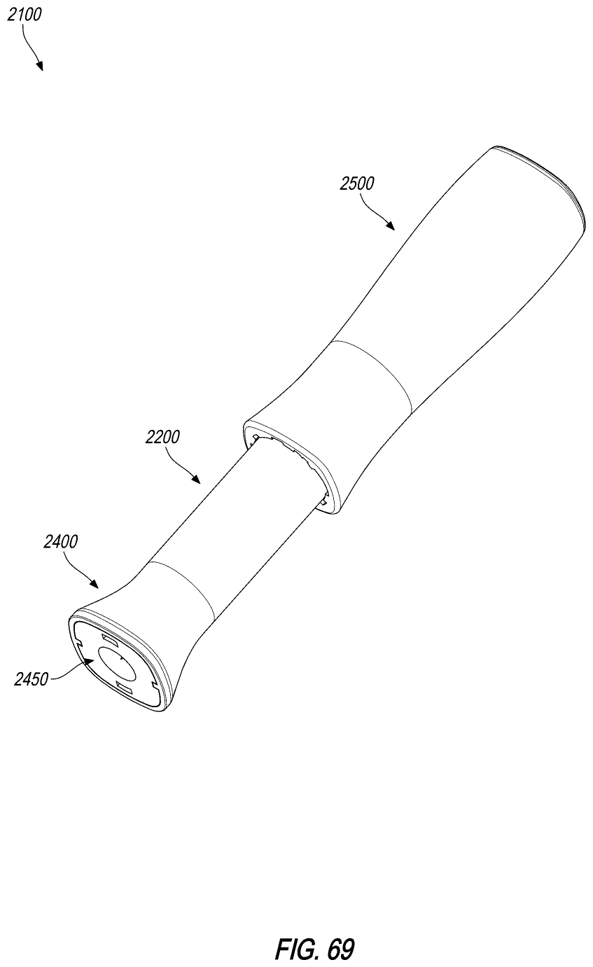

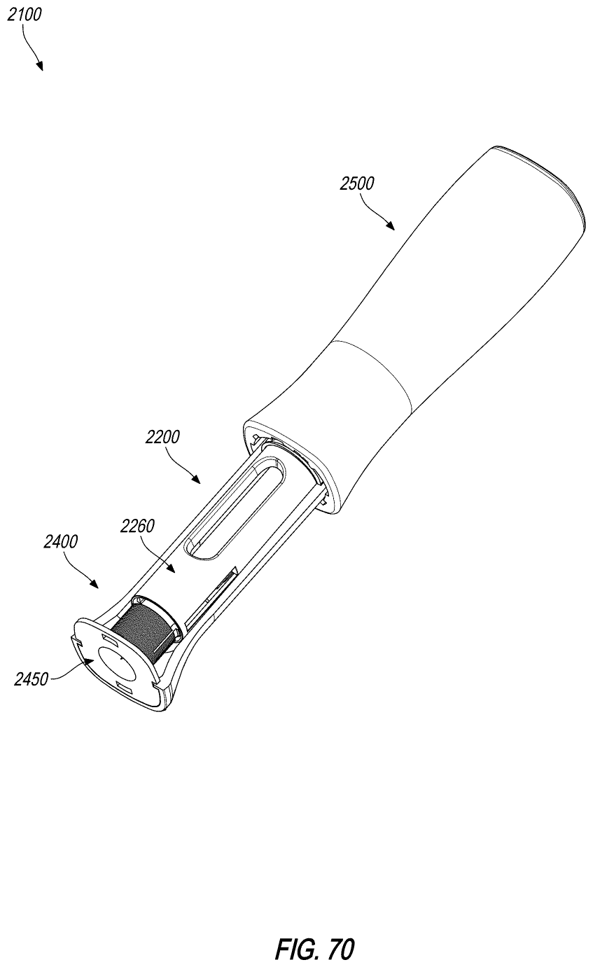

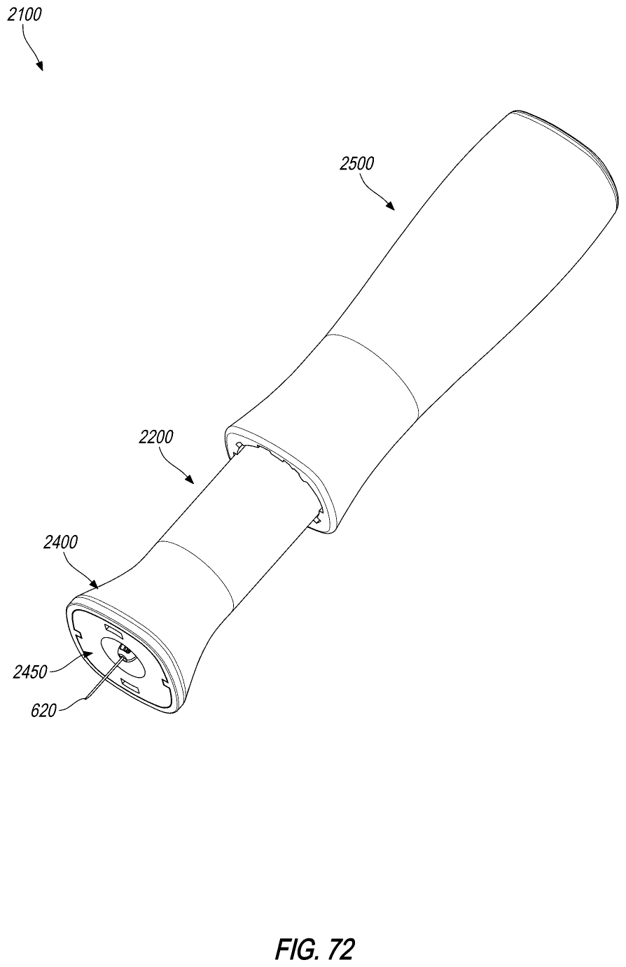

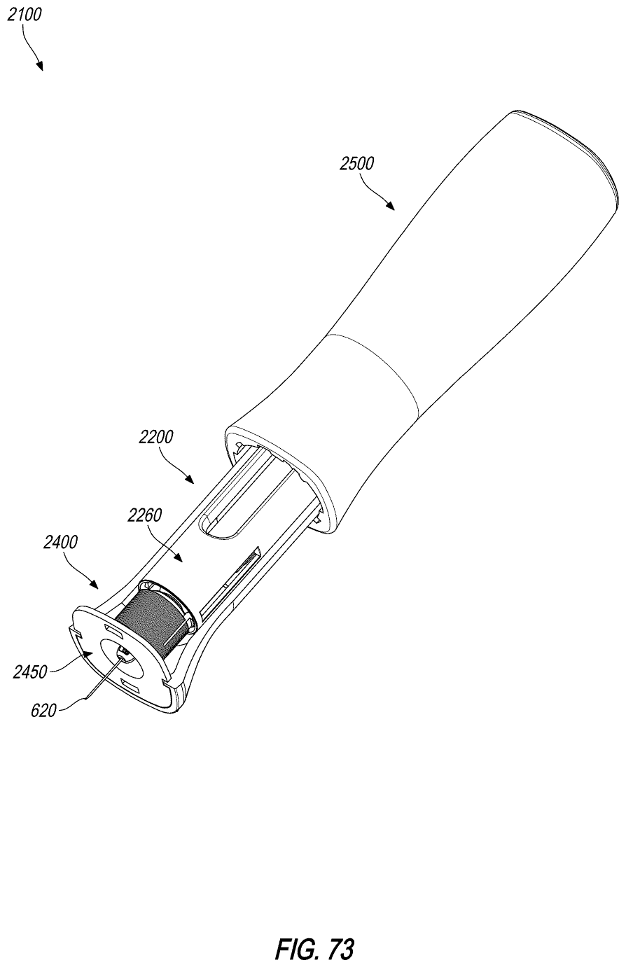

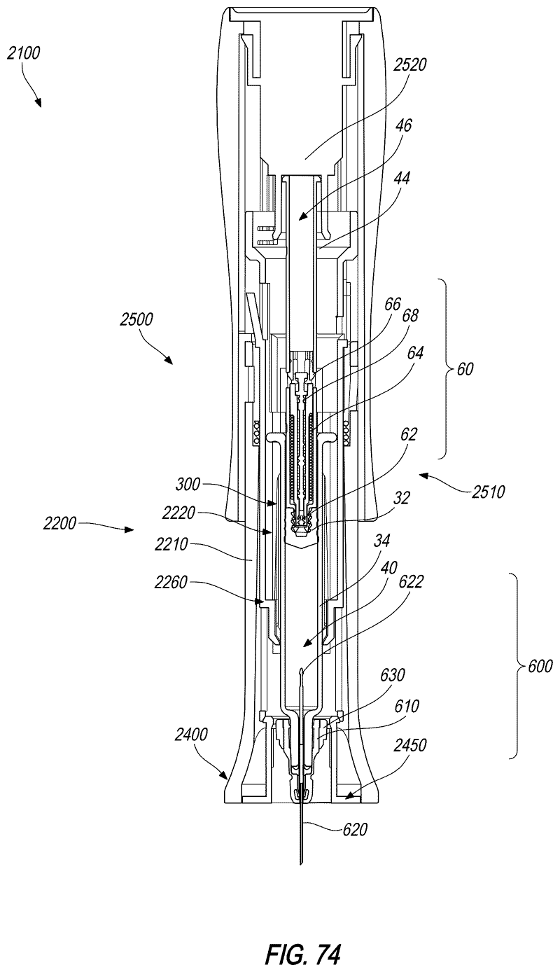

FIGS. 61A and 61B are side and detailed longitudinal cross-sectional views of the injection system (2100) in a ready-to-use configuration (see FIGS. 69 to 71), according to some embodiments. FIGS. 62A and 62B are side and detailed longitudinal cross-sectional views of the injection system (2100) in an extended configuration (see FIGS. 72 to 74, according to some embodiments. The handle (2500) is omitted from FIGS. 61A and 62A for clarity.

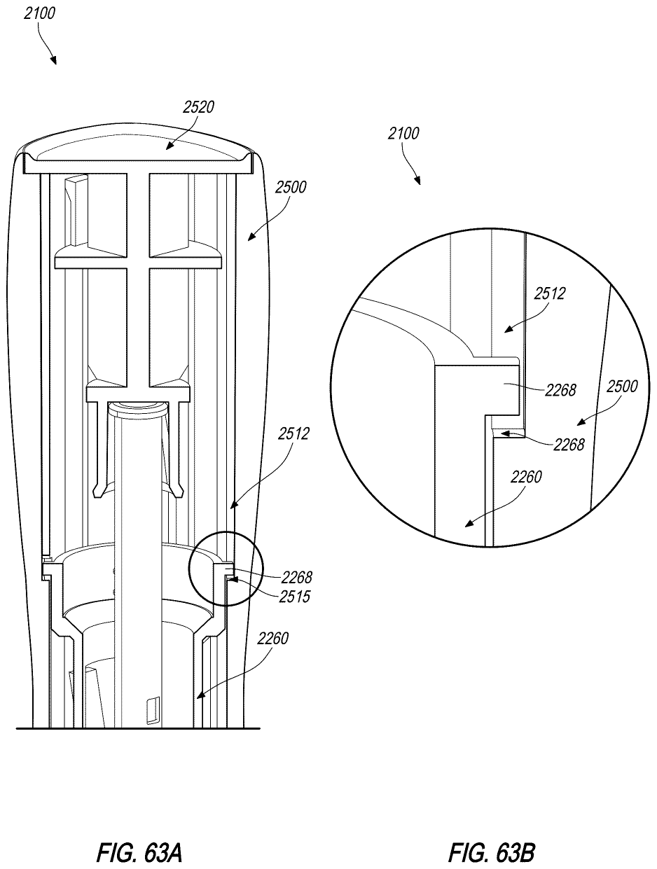

In the ready-to-use configuration before inserting the needle (620) depicted in FIGS. 61A, 61B, 63A, and 63B, the syringe housing (2260) and the rotating cuff (2270) coupled thereto are constrained proximally by a distally facing wall (2518) on an inner surface of the handle (2500) acting against the proximal surface of the radially extending block (2272) on the rotating cuff (2270) and distally by a proximally facing wall (2515) on an inner surface of the handle (2500) acting against the syringe housing radially outward extending tabs (2268). Further, the rotating cuff (2700) is held in its rotational/angular position by the distal tip of the angled block (2274) being disposed within the angled slot (2210) in the wall of the outer tube (2200).

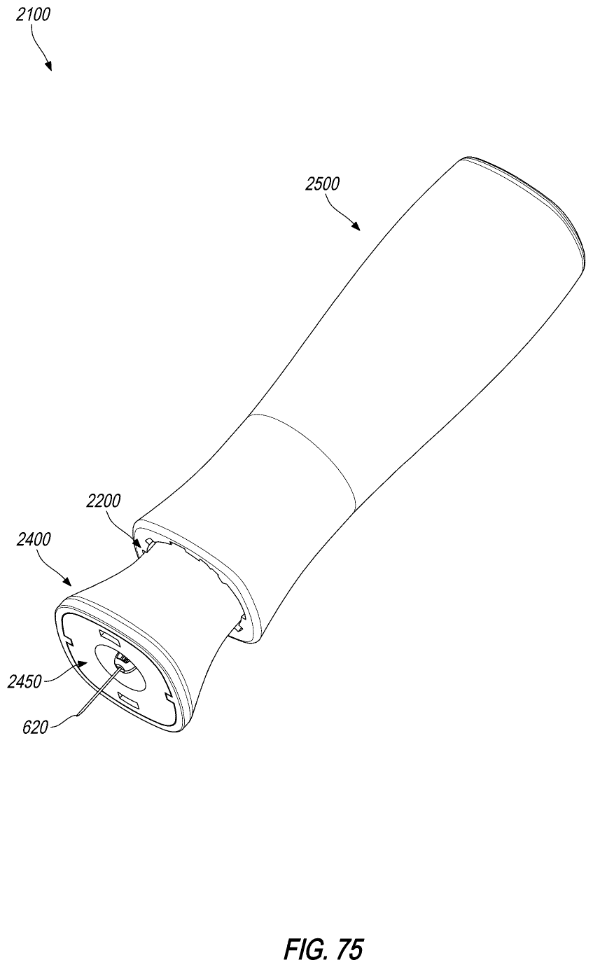

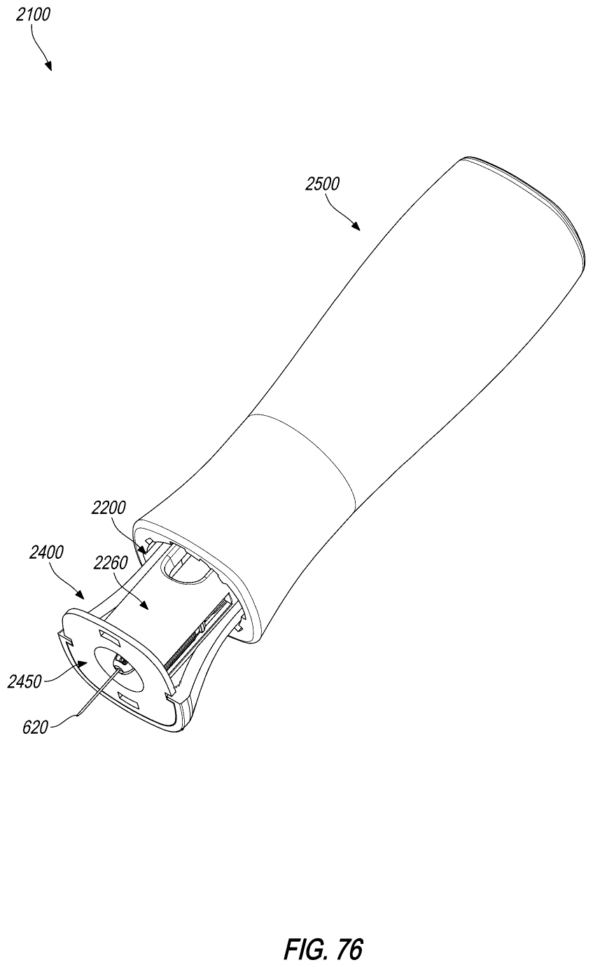

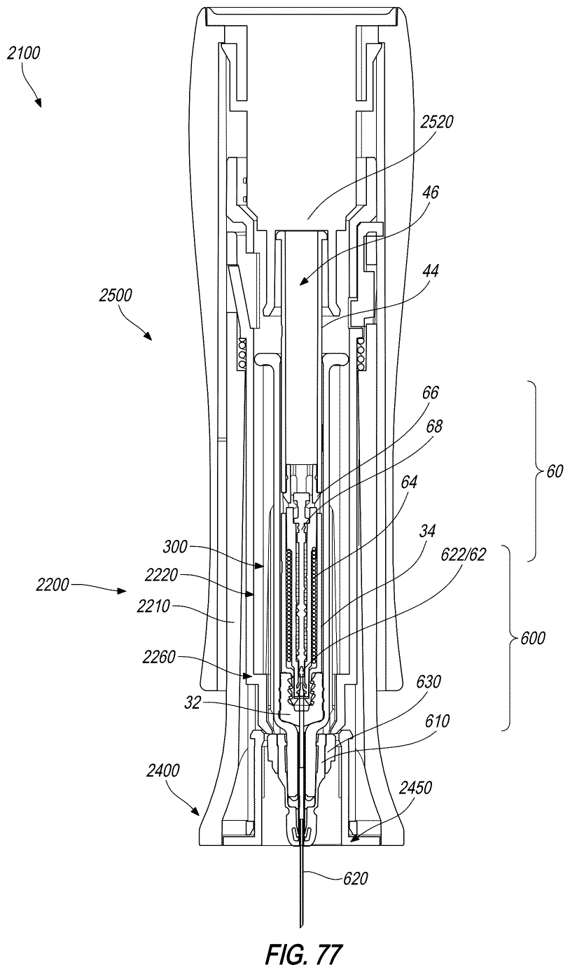

When a distally directed force is applied to the handle (2500) to insert the needle (620) into tissue of a patient, the distally directed force overcomes the force from the needle insertion spring (2290), and the angled block (2274) slides distally into the angled slot (2210) in the outer tube (2200), thereby advancing the needle (620) distally and rotating the rotating cuff (2700) around the syringe housing (2260). At the end of needle insertion/cuff rotation, the injection system (2100) has transformed from the ready-to-use configuration (FIGS. 61A and 61B) to the extended configuration depicted in FIGS. 62A and 62B. In the extended configuration, the radially extending block (2272) has rotated into a slot (2511) defined on the inner surface of the handle (2500) that allows the handle (2500) to move distally relative to the syringe housing (2260), thus allowing the user to inject the liquid from the syringe interior (40). Also, at the end of needle insertion/cuff rotation, the syringe housing anti-return latch (2264) on an outer surface of the syringe housing (2260) snaps into the anti-return window (2230) in the outer tube (2200) to prevent the injection member (300) and the syringe housing (2260) from moving proximally relative to the outer tube (2200). As described herein with reference to FIGS. 60A and 60B, during the transition from the ready-to-use configuration to the extended configuration, the needle insertion spring (2290) provides a similar resistance on the handle (2500) as the injection force during the transition between the extended configuration to the injected configuration (see FIGS. 75 to 77), thereby ensuring a smooth force profile over the needle insertion and injection processes.

FIGS. 63A and 63B are detailed and more detailed partially cutaway perspective views of the injection system (2100), according to some embodiments. When the injection system (2100) is in its assembled state shown in FIGS. 63A and 63B, the pair of syringe housing radially outward extending tabs (2268) at the proximal end of the syringe housing (2260) are supported distally by respective distal ends of a pair of syringe housing tab slots (2512) defined in an internal surface of the handle (2500). This prevents the handle (2500) from sliding proximally relative to the syringe housing (2260), which facilitates removal of the needle shield cap (2800) and the needle shield (760) attached thereto from the injection system (2100). The syringe housing tab slots (2512) allow free distal movement of the handle (2500) relative to the syringe housing (2260) to allow injection after the rotating cuff (2270) has been rotated into its injection position and the injection system (2100) has transformed from the ready-to-use configuration to the extended configuration. The pair of syringe housing radially outward extending tabs (2268) also cooperate with the respective syringe housing tab slots (2512) to prevent the handle (2500) from rotating in a first direction relative to the syringe housing (2260) while allowing the handle (2500) to move distally relative to the syringe housing (2260).

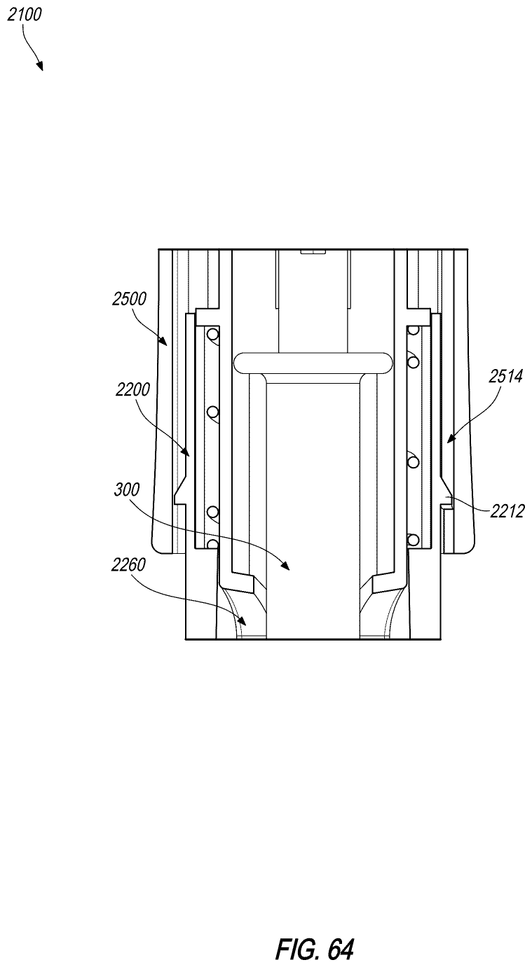

FIG. 64 is a detailed longitudinal cross-sectional view of the injection system (2100) at the proximal end of the syringe housing (2260), according to some embodiments. When the injection system (2100) is in its assembled state shown in FIG. 64, two outer tube radially outward extending tabs (2212) defined on an outer surface of the outer tube (2200) are supported distally by respective distal ends of a pair of outer tube tab slots (2514) defined in an internal surface of the handle (2500). This prevents the handle (2500) from sliding proximally relative to the outer tube (2200), which facilitates removal of the needle shield cap (2800) and the needle shield (760) attached thereto from the injection system (2100). The outer tube tab slots (2514) allow free distal movement of the handle (2500) relative to the outer tube (2200) for injection after the rotating cuff (2270) has been rotated into its injection position and the injection system (2100) has transformed from the ready-to-use configuration to the extended configuration

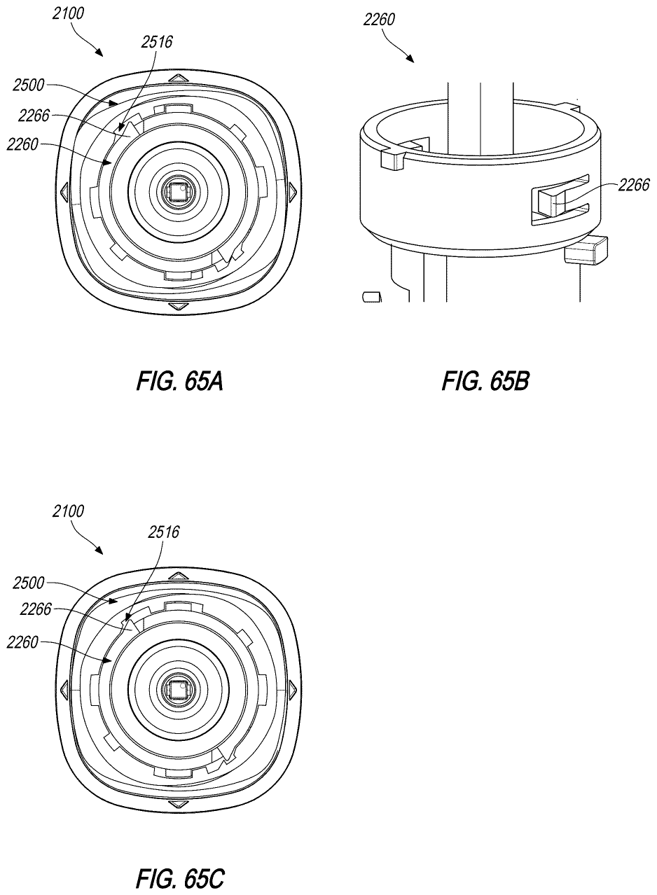

FIGS. 65A, 65B, and 65C are axial cross-sectional (FIGS. 65A and 65C) and detailed cutaway perspective (FIG. 65B) views of the injection system (2100) depicted in FIG. 46, according to some embodiments. When the injection system (2100) is in its assembled state shown in FIG. 65C, a pair of external housing latches (2266) on the syringe housing (2260) interact with a pair of respective syringe housing latch slots (2516) defined in an inner surface of the handle (2500) to prevent clockwise rotation of the syringe housing (2260) (i.e., in a second direction opposite of the first direction) within the handle (2500). This prevents the user from inadvertently twisting the handle (2500) into assembly mode during use. The external housing latches (2266) have an angled surface to allow the external housing latches (2266) to snap over slot walls in the respective syringe housing latch slots (2516) to switch from assembled (FIG. 65A) to use state (FIG. 65C).

FIGS. 66 to 68 are perspective, partial cutaway perspective, and longitudinal cross-sectional views of the injection system (2100) in a transport/storage configuration according to some embodiments. As described herein, the injection system (2100) has a housing member (2110), a syringe housing (2260), an injection member (300), a shroud (2450), and a needle shield handling member (2800). In the transport/storage configuration, the needle shield handling member (2800) and the needle shield (760) attached thereto are removably coupled to the shroud (2450) and the needle hub (610) respectively. As such, the needle shield (760) covers the sharp distal end of the needle (620).

As described herein with reference to FIGS. 52A and 52B, when the needle shield cap (2800) and the needle shield (760) are removably coupled to the shroud (2450) and the needle hub (610) respectively, the needle shield cap (760) interferes with the shroud arms (2452) to maintain a radial position of the shroud arms (2452) relative to the distal end of the syringe housing. This interference prevents the shroud arms (2452) from flexing radially inward, thereby preventing the syringe housing (2260) and the injection member (300) contained in the syringe housing (2260) from moving distally relative to the shroud (2450) and the outer tube (2200) until the needle shield cap (2800) and the needle shield (760) are removed from the needle hub (610) (see, e.g., FIG. 71). The shroud arms (2452) prevent the syringe housing (2260) and the injection member (300) from sliding distally during removal of the needle shield cap (2800) and the needle shield (760) to prevent the needle from being prematurely exposed.

FIGS. 69 to 71 are perspective, partial cutaway perspective, and longitudinal cross-sectional views of the injection system (2100) in a ready-to-use configuration according to some embodiments. The injection system (2100) can be transformed from the transport/storage configuration to the ready-to-use configuration by pulling the needle shield cap (2800) and the needle shield (760) distally away from the shroud (2450) and the rest of the injection system (2100). After the needle shield cap (2800) and the needle shield (760) are fully removed, the shroud arms (2452) are able to flex radially inward to into an open distal end of the syringe housing (2260) to allow the syringe housing (2260) and the injection member (300) to move distally to insert the needle (620) into tissue when a predetermined amount of force (configured to be provided by a user) is applied to the handle (2500).

Although the handle (2500) is free to move in the ready-to-use configuration, the needle insertion spring (2290) provides some resistance to this movement, thereby tuning the predetermined amount of force. The syringe housing (2260) and the rotating cuff (2270) coupled thereto are constrained proximally by a distally facing wall (2518) on an inner surface of the handle (2500) acting against the proximal surface of the radially extending block (2272) on the rotating cuff (2270) and distally by a proximally facing wall on an inner surface of the handle (2500) acting against the syringe housing radially outward extending tabs (2268), as shown in FIGS. 61A and 61B. Further, the rotating cuff (2700) is held in its rotational/angular position by the distal tip of the angled block (2274) being disposed within the angled slot (2210) in the wall of the outer tube (2200).

In the ready-to-use configuration, the needle obscuring member (2400) visually hides the sharp distal end of the needle (620) from the patient. The needle obscuring member (2400) may also tactilely hide the sharp distal end of the needle (620) from the patients by touching the skin of the patient at substantially or close to the same time that the sharp distal end of the needle (620) touches the skin of the patient. In a first step in an injection method, the injection system (2100) in the ready-to-use configuration is disposed in contact with a patient such that the needle obscuring member (2400) and the sharp distal end of the needle (620) is in contact with the skin of the patient.

FIGS. 72 to 74 are perspective, partial cutaway perspective, and longitudinal cross-sectional views of an injection system (2100) in an extended configuration according to some embodiments. The injection system (2100) can be transformed from the ready-to-use configuration to the extended configuration by placing the needle obscuring member (2400) against the skin of a patient and applying a distally directed force to the handle (2500), which is grasped in a hand of a user to first slide the injection member (300) distally in the outer tube (2200) to insert the needle (620) into the patient.

When the distally directed force is applied to the handle (2500) to insert the needle (620) into tissue of a patient, the distally directed force overcomes the force from the needle insertion spring (2290), and the angled block (2274) slides distally into the angled slot (2210) in the outer tube (2200), thereby advancing the needle (620) distally and rotating the rotating cuff (2700) around the syringe housing (2260). At the end of needle insertion/cuff rotation, the injection system (2100) has transformed from the ready-to-use configuration (FIGS. 61A and 61B) to the extended configuration (FIGS. 62A and 62B). In the extended configuration, the radially extending block (2272) has rotated into a slot (2511) defined on the inner surface of the handle (2500) that allows the handle (2500) to move distally relative to the syringe housing (2260), thus allowing the user to inject the liquid from the syringe interior (40). As described herein with reference to FIGS. 60A and 60B, during the transition from the ready-to-use configuration to the extended configuration, the needle insertion spring (2290) provides a similar resistance on the handle (2500) as the injection force during the transition between the extended configuration to the injected configuration thereby ensuring a smooth force profile over the needle insertion and injection processes.

FIGS. 75 to 77 are perspective, partial cutaway perspective, and longitudinal cross-sectional views of an injection system (2100) in an injection-complete configuration according to some embodiments. The injection system (2100) can be transformed from the extended configuration to the injection complete configuration by, with the needle (620) inserted into a patient, continuing to apply a distally directed force to the handle (2500). The continued application of the distally directed force moves the handle (2500), the plunger member (44) attached thereto, and the stopper member (32) attached to the plunger member (44) distally. Moving the stopper member (32) distally in the syringe interior (40) ejects a liquid medicine from the syringe interior (40) through the needle (620) and into the patients. Applying sufficient distally directed force to the handle (2500) to move the syringe member (32) to a distal end of the syringe body (34) completes the injection.

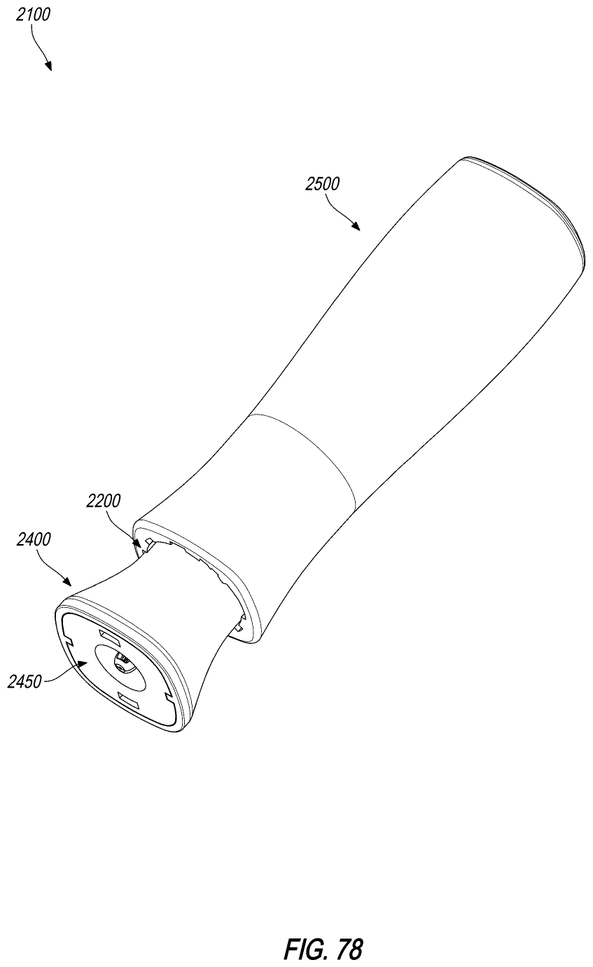

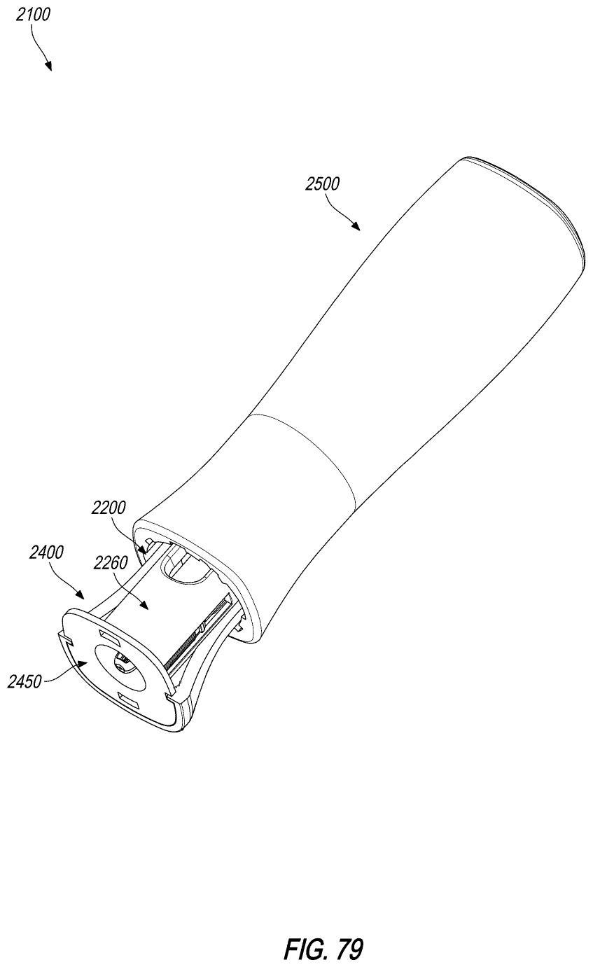

FIGS. 78 to 80 are perspective, partial cutaway perspective, and longitudinal cross-sectional views of an injection system (2100) in a needle-retracted/safe configuration according to some embodiments. The injection system (2100) can be transformed from the injection-complete configuration to the needle-retracted/safe configuration by, with the needle (620) inserted into a patient, continuing to apply a distally directed force to the handle (2500). Continued application of distally directed force to the handle (2500) both causes the proximal end member (622) of the needle (620) to be captured by/coupled to the needle coupling member (62) and moves the latch actuation member (68) to release the spring latch (66) thereby allowing the energy storage member/spring (64) to transform from the compressed/energized state to transform to an expanded/deenergized state. When transforming from the compressed state to the expanded state, the energy storage member/spring (64) pulls the needle coupling member (62) and the needle (620) attached thereto proximally into the injection system (2100) until the sharp distal end of the needle (620) is disposed in the needle hub (610) and a distal end of the syringe body (34), thereby rendering the injection system (2100) safe for disposal.

While the embodiments described above include single and dual chamber (safety) injection systems, the scope of the claims also include other multiple chamber injection systems, with or without safe injection systems. For multiple chamber safety injection systems with more than two chambers, more than two stopper members are inserted into an injection system body (e.g., syringe body, cartridge body, etc.) to define a corresponding number of chambers.

While the injection systems depicted and described herein include syringes with staked needles, the needle shield handling devices/telescoping members described herein can be used with cartridges, an auto injector, and injection systems with Luer connectors, etc. Any suitable manufacturing technique (e.g., molded unitary elements, molded pieces coupled together, laser cut, 3D laser printing, etching, etc.) may be used for the system and components described herein.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element-- irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. An injection system, comprising:
a housing member comprising
an outer tube defining a needle obscuring member at a distal end thereof,
a syringe housing disposed at least partially in the outer tube and defining a syringe housing interior, and
a handle slidably coupled to the outer tube and the syringe housing; and
an injection member disposed in the syringe housing interior, the injection member comprising
a syringe body having proximal and distal ends and defining a syringe interior,
a stopper member disposed in the syringe interior,
a plunger member coupled to the stopper member and configured to be manipulated to insert the stopper member distally in the syringe interior relative to the syringe body,
a needle hub assembly coupled to the syringe body at the distal end of the syringe body and comprising
a needle hub, and
a needle coupled to the needle hub,
wherein the needle obscuring member is configured to be disposed at least partially over the needle.

2. The injection system of claim 1, wherein the syringe housing defines a syringe housing latch adjacent a distal end of the syringe housing,
wherein the syringe housing defines a syringe housing rib adjacent a proximal end of the syringe housing, and
wherein the syringe housing latch and the syringe housing rib are configured to limit longitudinal movement of the injection member relative to the syringe housing.

3. The injection system of claim 1, further comprising a shroud coupled to the distal end of the outer tube,
wherein the shroud defines
a shroud arm at a proximal end of the shroud,
a shroud slot at a distal end of the shroud configured to align with a tab defined by the outer tube at a distal end of the outer tube, and
a shroud opening at the distal end of the shroud.

4. The injection system of claim 3, wherein the injection member further comprises a needle shield removably coupled to the needle hub and covering the needle, and
wherein the needle shield defines
a needle shield distal opening at a distal end of the needle shield, and
a needle shield longitudinal slot along a radially outward surface of the needle shield.

5. The injection system of claim 4, further comprising a needle shield cap,
wherein the needle shield cap defines
a needle shield cap latch configured to cooperate with the needle shield distal opening to couple the needle shield cap to the needle shield,
a needle shield cap guide column configured to cooperate with the needle shield longitudinal slot to couple the needle shield cap to the needle shield, and
a needle shield cap detent configured to cooperate with the shroud opening to couple the needle shield cap to the shroud.

6. The injection system of claim 5, wherein, when the needle shield cap and the needle shield are removably coupled to the needle hub, the needle shield cap interferes with the shroud arm to maintain a radial position of the shroud arm relative to a distal end of the syringe housing, thereby preventing the syringe housing and the injection member contained in the syringe housing from moving distally relative to the shroud and the outer tube until the needle shield cap and the needle shield are removed from the needle hub.

7. The injection system of claim 1, further comprising a needle insertion spring disposed around the syringe housing and configured to provide a needle insertion resistance to distal movement of the syringe housing relative to the outer tube.

8. The injection system of claim 7, wherein the needle insertion resistance is substantially equal to a stopper advancement resistance to distal advancement of the stopper member relative to the syringe body.

9. The injection system of claim 1, further comprising a rotating cuff disposed around a proximal portion of the syringe housing and translationally coupled to the syringe housing,
wherein the rotating cuff defines
a radially extending block at a proximal end of the rotating cuff, and
an angled block at a distal end of the rotating cuff.

10. The injection system of claim 9, wherein the handle defines a distally facing wall configured to cooperate with the radially extending block to prevent distal movement of the handle relative to the rotating cuff and the syringe housing coupled thereto before the syringe housing and the rotating cuff coupled thereto move distally relative to the outer tube to prevent premature ejection of a liquid from the syringe interior.

11. The injection system of claim 9, wherein the outer tube defines an angled slot at a proximal end of the outer tube configured to cooperate with the angled block to control rotation of the rotating cuff relative to the outer tube and the handle, and
wherein moving the syringe housing and the rotating cuff coupled thereto distally relative to the outer tube rotates the rotating cuff relative to the syringe housing, the outer tube and the handle.

12. The injection system of claim 11, wherein rotating the rotating cuff relative to the syringe housing, the outer tube and the handle rotates the radially extending block away from the distally facing wall and into a slot defined on an inner surface of the handle to allow the handle to move distally relative to the rotating cuff and the syringe housing coupled thereto to eject a liquid from the syringe interior.

13. The injection system of claim 1, wherein the syringe housing defines a syringe housing anti-return latch,
wherein the outer tube defines an anti-return window configured to cooperate with the syringe housing anti-return latch to prevent the syringe housing from moving proximally relative to the outer tube after the syringe housing moves distally relative to the outer tube to a distal limit of travel of the syringe housing.

14. The injection system of claim 1, wherein the syringe housing defines a syringe housing radially outward extending tab configured to cooperate with a syringe housing tab slot defined in an inner surface of the handle to prevent the handle from rotating in a first direction or moving proximally relative to the syringe housing while allowing the handle to move distally relative to the syringe housing after rotation of the rotating cuff relative to the handle.

15. The injection system of claim 1, wherein the outer tube defines an outer tube radially outward extending tab configured to cooperate with an outer tube tab slot defined in an inner surface of the handle to prevent the handle from moving proximally relative to the outer tube while allowing the handle to move distally relative to the outer tube.

16. The injection system of claim 1, wherein the syringe housing defines a syringe housing external housing latch configured to cooperate with a syringe housing latch slot defined in an inner surface of the handle to prevent the handle from rotating in a second direction relative to the syringe housing after assembly.

17. The injection system of claim 1, further comprising a handle cap disposed at a proximal end of the handle,
wherein the handle cap defines a handle cap latch configured to cooperate with a proximal end wall defined on an inner surface of the handle to prevent the handle cap from moving proximally relative to the handle.

18. (canceled)

19. The injection system of claim 1, wherein the handle cap defines a plunger member arm configured to cooperate with a distally facing surface at a proximal end of the plunger member to prevent the handle from moving proximally relative to the plunger member.

20. An injection system, comprising:
a housing member comprising a housing body defining a housing interior; and
an injection member disposed in the housing interior, the injection member comprising
a syringe body having proximal and distal ends and defining a syringe interior,
a stopper member disposed in the syringe interior,
a plunger member coupled to the stopper member and configured to be manipulated to insert the stopper member distally in the syringe interior relative to the syringe body,
a needle hub assembly coupled to the syringe body at the distal end thereof and comprising
a needle hub, and
a needle coupled to the needle hub,
wherein the housing member comprises a needle obscuring member slidably coupled to the housing body, and
wherein the needle obscuring member is configured to be disposed at least partially over the needle.

21.-30. (canceled)

31. An injection system, comprising:
a housing member comprising a housing body defining a housing interior;
an injection member disposed in the housing interior, the injection member comprising
a syringe body having proximal and distal ends and defining a syringe interior,
a stopper member disposed in the syringe interior,
a plunger member coupled to the stopper member and configured to be manipulated to insert the stopper member distally in the syringe interior relative to the syringe body,
a needle hub assembly coupled to the syringe body at the distal end thereof and comprising
a needle hub, and
a needle coupled to the needle hub; and
a plunger manipulation member having an open distal end and a plunger interface coupled to a proximal end of the plunger member,
wherein the housing member comprises a needle obscuring member coupled to the housing body, and
wherein the needle obscuring member is configured to be disposed at least partially over the needle.

32.-37. (canceled)
